(19) United States
(12) Patent Application Publication
(10) Pub. No.: US 2025/0381395 A1
(43) Pub. Date: Dec. 18, 2025

(54) METHODS AND DEVICES FOR CUSTOMIZATION AND PERSONALIZATION OF ENERGY-BASED AESTHETIC TREATMENTS

(71) Applicant: Like Zeng, Pleasanton, CA (US)

(72) Inventor: Like Zeng, Pleasanton, CA (US)

(21) Appl. No.: 19/236,716

(22) Filed: Jun. 12, 2025

Related U.S. Application Data
(60) Provisional application No. 63/659,089, filed on Jun. 12, 2024.
(60) Provisional application No. 63/700,680, filed on Sep. 28, 2024.

(51) Int. Cl. A61N 1/32; A61N 1/02; A61N 1/36; G16H 40/63

(52) U.S. Cl. CPC A61N 1/328; A61N 1/025; A61N 1/36031; A61N 1/36034; G16H 40/63

(57) ABSTRACT

A method of customizing an energy-based aesthetic treatment includes obtaining image data identifying a treatment area and a tissue-contacting part of a treatment device. The method further includes selecting an anatomical classification criterion based on a type of energy modality used for said treatment. The method further includes determining, by analyzing the image data using the anatomical classification criterion, a plurality of treatment sub-areas that are differentiated based on a measurable or observable property of an anatomical characteristic which varies across regions of tissue in the treatment area. The method further includes determining, while said treatment is being performed, a current treatment sub-area of the treatment area that is actively being treated or targeted for treatment, and adjusting one or more treatment settings of the treatment device based on the measurable or observable property of the anatomic characteristic of a region of tissue in the current treatment sub-area.

### RELATED APPLICATION

This application claims the benefit of U.S. Provisional Patent Application No. 63/700,680, filed on Sep. 28, 2024, which claims the benefit of U.S. Provisional Patent Application No. 63/659,089, filed on Jun. 12, 2024, the contents of each of which is incorporated herein by reference in their entireties.

### FIELD OF THE INVENTION

The present invention generally relates to devices and methods for customizing and personalizing energy-based aesthetic treatments.

### BACKGROUND OF THE INVENTION

Energy-based aesthetic treatment devices are widely used in both professional clinical settings and for personal use at home. These treatment devices are designed to improve aesthetic conditions by delivering energy to targeted tissue areas. The energy delivered can be radiofrequency (RF) energy, ultrasound energy, intense pulsed light (IPL) energy, laser energy, low level light therapy energy, and/or the like. The energy-based treatments can be used to tighten loose skin, reduce wrinkles, remove pigmentation, remove fatty tissue, and/or other skin rejuvenation goals. Typically, treatment devices come with pre-programmed treatment settings and/or treatment plans that offer only a limited number of choices for operators to choose from.

One disadvantage of conventional treatment devices is the treatment settings are not customized to the unique physical and physiological properties of the tissue. The physical and physiological properties of the tissue vary greatly between different anatomical areas. For instance, facial and neck treatments often involve treating a number of different parts of a patient's face and check, including the forehead, periorbital area, eye, area under the eye, nose, cheek, lips, chin, submental and lateral neck. In these treatments, different areas of the face and/or neck have different pain thresholds. Using a single treatment intensity setting might be suitable for one area but for another area could trigger nociceptors of the patient, thereby causing pain, discomfort, and/or harm. Although some devices offer several treatment intensity settings (e.g., low, medium, high power levels) for operators to choose from, it is often impractical or tedious for operators to adjust these settings continuously during a treatment session as they treat different areas. This issue becomes more pronounced when the device is used by laypersons at home.

Another disadvantage is the reliance on the operator to ensure the treatment quality is maintained throughout the energy-based aesthetic treatments. Incorrect placement of the tissue-contacting part of the energy-based aesthetic treatment device can result in significant variations in energy output, posing not only effectiveness concerns but safety risks. This challenge is especially problematic for laypersons using over-the-counter devices at home, where distractions are common. Incorrect usage can lead to treating risk areas, such as using monopolar radiofrequency devices near the eyes, which is hazardous. Light-based devices like IPL, laser, or lower level light therapy treatments require the operator to avoid the eye area to prevent harm. Ultrasound based devices require the operator to constantly check if the ultrasound transducer has adequate coupling. This demand for constant vigilance is burdensome for physicians and nearly unmanageable for non-professional users in home environments.

Yet another disadvantage is the current state of art energy based aesthetic devices lack the capability of monitoring immediate tissue responses. The treated tissue often exhibits immediate skin reactions, including but not limited to redness (or erythema) and swelling (or edema). These reactions can serve as critical indicators of the skin's response to the treatment, with excessive redness and edema typically signaling over-treatment. In such cases, it is necessary to either stop the treatment or reduce the treatment intensity to avoid post-treatment adverse effects. However, current state-of-the-art devices lack the capability to monitor these immediate skin reactions in real-time. Instead, they rely on operators or physicians to visually assess and judge whether these reactions are within normal range. This reliance on human judgment requires the operator to remain constantly vigilant throughout the treatment process, which is both burdensome and prone to human error.

Yet another notable deficiency of current energy-based aesthetic devices is their inability to provide personalized treatment planning. The physical, physiological, anatomical, and pathological properties of tissue can vary significantly among patients, necessitating tailored treatment plans to optimize results. In professional clinical settings, where pre-treatment consultations are available, such personalized treatment plan can be developed. However, this process increases the time and labor required from professionals during consultations. In at-home or over-the-counter settings, where professional guidance is typically unavailable, the absence of a personalized treatment plan becomes even more pronounced. This limitation not only hinders treatment effectiveness but also raises concerns regarding safety and patient satisfaction, as users may not achieve the desired outcomes without individualized planning.

Yet another significant deficiency of current energy-based aesthetic devices is their inability to provide real-time feedback on the treatment techniques used by their respective operators. This limitation is particularly pronounced in at-home settings, where individual users exhibit varied treatment styles. For instance, some users may move the device too slowly, while others may move it too quickly, both of which compromise optimal energy delivery to the tissue. Furthermore, discrepancies in the treatment area—where some users may inadvertently treat a larger or smaller area than intended—can adversely affect aesthetic outcomes and raise safety concerns. Additionally, the potential for user distraction in home environments exacerbates these issues, leading to deviations from recommended treatment protocols. This lack of feedback mechanisms undermines the efficacy and safety of energy-based aesthetic treatments.

In light of these disadvantages, it is evident that there is a need for improved energy-based aesthetic treatment devices and methods that offer customization and personalization to ensure treatment safety, comfort, effectiveness, and ease of use.

### SUMMARY OF THE INVENTION

In an aspect of the invention, a method of customizing an energy-based aesthetic treatment is provided. The method includes obtaining, by a treatment device, image data identifying a treatment area and a tissue-contacting part of the treatment device. Image data may, for example, be obtained by receiving the image data from an image acquisition module of the treatment device. The method further includes selecting, by the treatment device, an anatomical classification criterion based on a type of energy modality used for the energy-based aesthetic treatment. The anatomical classification criterion is an anatomical characteristic with a measurable or observable property that differs across regions of tissue in the treatment area. The method further includes determining, by the treatment device and by analyzing the image data using the anatomical classification criterion, a plurality of treatment sub-areas within the treatment area. The treatment sub-areas are differentiated based on the measurable or observable property of the anatomical characteristic which varies across the regions of tissue in the treatment area. While the energy-based aesthetic treatment is being performed the method further includes determining, by the treatment device, a current treatment sub-area of the treatment area that is actively being treated or targeted for treatment. The current treatment sub-area is determined based on a position of the tissue-contacting part of the treatment device relative to one of the plurality of treatment sub-areas. The method further includes adjusting, by the treatment device, one or more treatment settings during the energy-based aesthetic treatment based on the measurable or observable property of the anatomic characteristic of a region of tissue in the current treatment sub-area.

In an embodiment of the invention, the method further includes determining that the tissue-contacting part of the treatment device at least partially overlaps with a risk area. The method further includes performing one or more corrective actions including at least one of: adjusting at least one treatment setting of the one or more treatment settings, and generating and providing a warning or recommendation for display on a user interface accessible by an operator of the treatment device.

In another embodiment of the invention, the method further includes determining that the tissue-contacting part of the treatment device is outside of the current treatment sub-area. The method further includes performing one or more corrective actions including at least one of: adjusting at least one treatment setting of the one or more treatment settings, and generating and providing a warning or recommendation for display on a user interface accessible by an operator of the treatment device.

In another embodiment of the invention, wherein the anatomical classification criterion is one of: a skin thickness, a depth of a bone relative to a skin surface, a depth of a muscle or a nerve fiber relative to the skin surface, an orientation of the muscle or the nerve fiber, or a skin condition.

In another embodiment of the invention, the anatomical classification criterion selected is a skin thickness. The plurality of treatment sub-areas are determined to be regions of tissue containing different skin thicknesses. In this embodiment, adjusting the one or more treatment settings includes adjusting a treatment intensity level based on the skin thickness identified in the region of tissue included in the current treatment sub-area.

In another embodiment of the invention, the anatomical classification criterion selected is a depth of a bone relative to a skin surface. The plurality of treatment sub-areas are determined to be regions of tissue containing bones located at different depths relative to the skin surface. In this embodiment, adjusting the one or more treatment settings includes adjusting a focal depth level based on the depth of the bone in the region of tissue included in the current treatment sub-area.

In another embodiment of the invention, the anatomical classification criterion selected is a depth of a muscle or a nerve fiber relative to a skin surface. The plurality of treatment sub-areas are determined to be regions of tissue containing muscles or nerve fibers located at different depths. In this embodiment, adjusting the one or more treatment settings includes adjusting an amplitude of an electrical waveform based on the depth of the muscle or the nerve fiber in the region of tissue included in the current treatment sub-area.

In another embodiment of the invention, the anatomical classification criterion selected is an orientation of a muscle or a nerve fiber. The plurality of treatment sub-areas are determined to be regions of tissue containing muscles or nerve fibers oriented in different directions. In this embodiment, adjusting the one or more treatment settings includes adjusting a direction of electrical current delivery to be aligned or substantially aligned with the orientation of the muscle or the nerve fiber.

In another embodiment of the invention, the anatomical classification criterion selected is a skin condition. The plurality of treatment sub-areas are determined to be regions of tissue corresponding to different skin conditions. In this embodiment, adjusting the one or more treatment settings includes adjusting one or more light wavelength settings based on the skin condition found in the region of tissue included in the current treatment sub-area.

In another embodiment of the invention, the treatment device is configured to deliver at least two different types of energy. In this embodiment, selecting the anatomical classification criterion includes: selecting the anatomical classification criterion based on a first type of energy modality used for the energy-based aesthetic treatment, and further selecting another anatomical classification criterion based on a second type of energy modality used for the energy-based aesthetic treatment. In this embodiment, determining the plurality of treatment sub-areas includes: determining a first set of treatment sub-areas as regions of tissue containing the measurable or observable property of the anatomical classification criterion, and determining a second set of treatment sub-areas as regions of tissue containing a measurable or observable property of the other anatomical classification criterion. In this embodiment, the method further includes delivering a first type of energy, a second type of energy, or both the first and second type of energy, at a moment in time, based on whether the tissue-contacting part of the treatment device is within one or both of: the first set of treatment sub-areas, and the second set of treatment sub-areas.

In an aspect of the invention, a treatment device is provided. The treatment device includes one or more cameras configured to capture image data identifying a treatment area and a tissue-contacting part of the treatment device, an energy emission component configured to deliver energy as part of an energy-based aesthetic treatment, one or more memories; and one or more processors, communicatively coupled to the one or more cameras and operatively coupled to the energy emission component and the one or more memories. The one or more processors are to receive the image data from the one or more cameras. The one or more processors are further to select an anatomical classification criterion based on a type of energy modality used for the energy-based aesthetic treatment. The anatomical classification criterion is an anatomical characteristic with a measurable or observable property that differs across regions of tissue in the treatment area. The one or more processors are further to determine, by analyzing the image data using the anatomical classification criterion, a plurality of treatment sub-areas within the treatment area. The treatment sub-areas are differentiated based on the measurable or observable property of the anatomical characteristic which varies across the regions of tissue in the treatment area. While the energy-based aesthetic treatment is being performed, the one or more processors are further to determine a current treatment sub-area of the treatment area that is actively being treated or targeted for treatment. The current treatment sub-area is determined based on a position of the tissue-contacting part of the treatment device relative to one of the plurality of treatment sub-areas. The one or more processors are further to adjust one or more treatment settings during the energy-based aesthetic treatment based on the measurable or observable property of the anatomic characteristic of a region of tissue in the current treatment sub-are, adjustment of the one or more treatment settings influencing the energy delivered by the energy emission component.

In an embodiment of the invention, the one or more processors are further to determine that the treatment area contains a risk area. The one or more processors are further to determine that the current treatment sub-area is within the risk area. The one or more processors are further to perform one or more corrective actions including at least one of: adjust at least one treatment setting of the one or more treatment settings, and generate and provide a warning or recommendation for display on a user interface accessible by an operator of the treatment device.

In another embodiment of the invention, the one or more processors are further to determine that the current treatment sub-area is outside of an intended treatment sub-area. The one or more processors are further to perform one or more corrective actions including at least one of: adjust at least one treatment setting of the one or more treatment settings, and generate and provide a warning or recommendation for display on a user interface accessible by an operator of the treatment device.

In another embodiment of the invention, the anatomical classification criterion is one of: a skin thickness, a depth of a bone relative to a skin surface, a depth of a muscle or a nerve fiber relative to the skin surface, an orientation of the muscle or the nerve fiber, or a skin condition.

In another embodiment of the invention, the anatomical classification criterion selected is a skin thickness. The plurality of treatment sub-areas are determined to be regions of tissue containing different skin thicknesses. In this embodiment, the one or more processors, when adjusting the one or more treatment settings, are to adjust a treatment intensity level based on the skin thickness identified in the region of tissue included in the current treatment sub-area.

In another embodiment of the invention, the anatomical classification criterion selected is a depth of a bone relative to a skin surface. The plurality of treatment sub-areas are determined to be regions of tissue containing bones located at different depths relative to the skin surface. In this embodiment, the one or more processors, when adjusting the one or more treatment settings, are to adjust a focal depth level based on the depth of the bone in the region of tissue included in the current treatment sub-area.

In another embodiment of the invention, the anatomical classification criterion selected is a depth of a muscle or a nerve fiber relative to a skin surface. The plurality of treatment sub-areas are determined to be regions of tissue containing muscles or nerve fibers located at different depths. In this embodiment, the one or more processors, when adjusting the one or more treatment settings, are to adjust an amplitude of an electrical waveform based on the depth of the muscle or the nerve fiber in the region of tissue included in the current treatment sub-area.

In another embodiment of the invention, the anatomical classification criterion selected is an orientation of a muscle or a nerve fiber relative to a known reference orientation. The plurality of treatment sub-areas are determined to be regions of tissue containing muscles or nerve fibers oriented in different directions. In this embodiment, the one or more processors, when adjusting the one or more treatment settings, are to adjust a direction of electrical current delivery to be aligned or substantially aligned with the orientation of the muscle or the nerve fiber.

In another embodiment of the invention, the anatomical classification criterion selected is a skin condition. The plurality of treatment sub-areas are determined to be regions of tissue corresponding to different skin conditions. In this embodiment, the one or more processors, when adjusting the one or more treatment settings, are to: adjust one or more light wavelength settings based on the skin condition found in the region of tissue included in the current treatment sub-area.

In another embodiment of the invention, the treatment device is configured to deliver at least two different types of energy. In this embodiment, the one or more processors, when selecting the anatomical classification criterion, are to select the anatomical classification criterion based on a first type of energy modality used for the energy-based aesthetic treatment, and select another anatomical classification criterion based on a second type of energy modality used for the energy-based aesthetic treatment. In this embodiment, the one or more processors, when determining the plurality of treatment sub-areas, are to determine a first set of treatment sub-areas as regions of tissue containing the measurable or observable property of the anatomical classification criterion, and determine a second set of treatment sub-areas as regions of tissue containing a measurable or observable property of the other anatomical classification criterion. In this embodiment, the one or more processors are further to deliver a first type of energy, a second type of energy, or both the first and second type of energy, at a moment in time, based on whether the tissue-contacting part of the treatment device is within one or both of: the first set of treatment sub-areas and the second set of treatment sub-areas.

### SUMMARY OF THE FIGURES

The accompanying drawings are included to provide a further understanding and are incorporated in and constitute a part of this specification. The drawings illustrate one or more embodiment(s), and together with the description serve to explain principles and operation of the various embodiments. Features and attributes associated with any of the embodiments shown or described may be applied to other embodiments shown, described, or appreciated based on this disclosure.

FIG. 1 is a diagram of a treatment device according to the principles of the present disclosure.

FIG. 2 is a diagram of a treatment area that includes key point delineated treatment sub-areas that are used for customizing an energy-based aesthetic treatment.

FIG. 3 is a diagram of a treatment area that includes the key point delineated treatment sub-areas and a key point delineated tissue-contacting part of the treatment device.

FIG. 4A is a diagram showing an example where the tissue-contacting part of the treatment device is not fully enclosed within the treatment sub-area.

FIG. 4B is a diagram showing an example where the tissue-contacting part of the treatment device overlaps with a risk area.

FIG. 5 is a diagram showing an example where a treatment sub-area contains muscle or nerve fibers oriented along a longitudinal direction a or a longitudinal direction b.

FIG. 6 is a diagram showing an example of a treatment sub-area associated with a skin condition.

FIG. 7 is a diagram showing an example of a first treatment sub-area associated with a skin thickness and a second treatment sub-area associated with a skin condition, where the second treatment sub-area is located within the first treatment sub-area.

FIG. 8A is a diagram showing an area that the tissue-contacting part of the treatment device has traversed at a time t.

FIG. 8B is a diagram showing an area that the tissue-contacting part of the treatment device has traversed during a first time window.

FIG. 8C is a diagram showing an area that the tissue-contacting part of the treatment device has traversed during a second time window.

FIG. 8D is a diagram showing an area that the tissue-contacting part of the treatment device has traversed during a third time window.

FIG. 8E is a diagram showing an area that the tissue-contacting part of the treatment device has traversed during a fourth time window.

FIG. 8F is a diagram showing an area that the tissue-contacting part of the treatment device has traversed during a fifth time window.

### DETAILED DESCRIPTION OF THE INVENTION

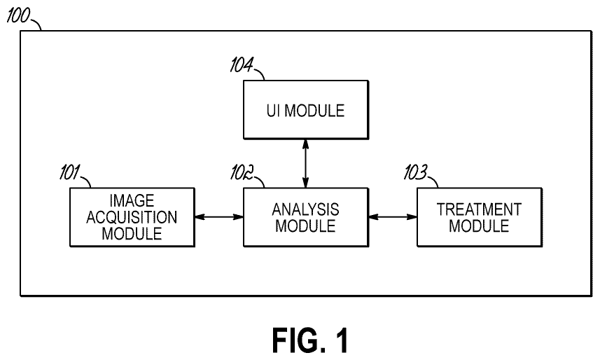

As shown in FIG. 1, the energy-based aesthetic treatment device 100 (sometimes referred to herein as the treatment device 100) may include an image acquisition module 101, an analysis module 102, a treatment module 103, and a user interface (UI) module 104.

The image acquisition module 101 within the treatment device 100 facilitates image capture for treatment analysis and optimization. The image acquisition module 101 captures treatment-related images and transmits the images to the analysis module 102 for processing and analysis. Communication between modules of the treatment device 100, including transmission of image data from the image acquisition module 101 to the analysis module 102, may be implemented using a bus and/or other circuitry and/or communication interfaces known in the art.

In some embodiments, the image acquisition module 101 may include visible light digital camera(s). This type of camera often employs either CCD (charge-coupled device) or CMOS (complementary metal-oxide-semiconductor) sensor technology to convert incoming light into digital signals, which are then processed to produce digital images. Visible light digital cameras are cost effective and widely used in electronics.

In some embodiments, infrared thermography camera(s) may be integrated into the image acquisition module 101 due to their ability to capture thermal images, offering insights into tissue temperature variations and aiding in the optimization of treatment settings.

In some embodiments, the image acquisition module 101 may also include microscopic cameras for capturing high-resolution images at a microscopic level, enabling detailed analysis of tissue structures and imperfections for customizing and personalizing the energy-based aesthetic treatments. The captured microscopic high-resolution images may be individually analyzed by the analysis module 102. The captured microscopic high-resolution images may be analyzed by the analysis module 102.

In some embodiments, the image acquisition module 101 may include fluorescent imaging capabilities. Fluorescent imaging leverages the natural or induced fluorescence of biological molecules to visualize different structures and conditions in the skin. When a specific wavelength of light (excitation wavelength) is directed at the skin, certain molecules absorb this energy and re-emit it at a longer wavelength (emission wavelength). This emitted light can be captured using cameras and analyzed to reveal information about the skin's structure and condition. To detect sebum, ultraviolet (UV) light with an excitation wavelength of approximately 370-380 nm may be used. Sebum contains lipids that fluoresce when exposed to this UV light, emitting light at wavelengths greater than 500 nm. This emission typically appears as green or yellow fluorescence, making sebum visible against the non-fluorescing background of the skin. To detect porphyrins, which are associated with acne due to their production by Propionibacterium acnes bacteria, near-UV light with an excitation wavelength of 400-410 nm may be used. When exposed to this light, porphyrins fluoresce red, emitting light at wavelengths between 600-650 nm. This red fluorescence highlights areas of bacterial activity, aiding in the identification and treatment of acne. To detect pigmentation, particularly due to melanin, UV light within the 320-400 nm range may be used. Pigmentation emits light in a broad spectrum, often around 420-560 nm. This emission allows for the visualization of pigmented areas, facilitating the assessment of pigmentation disorders. To detect structural proteins collagen and elastin in the skin, UVA light with an excitation wavelength of 335-365 nm may be used, these proteins fluoresce, emitting blue to green light within the 400-500 nm range. This fluorescence helps in assessing the structural integrity and health of the dermal layer. To detect lipofuscin, known as the aging pigment, UV light with an excitation wavelength of 340-380 nm may be used. Lipofuscin fluoresces yellow to orange, emitting light within the 520-620 nm range. Its presence can indicate oxidative stress and aging in the skin, providing valuable information for anti-aging treatments.

In some embodiments, the image acquisition module 101 may include multi-spectral imaging capabilities. Multi-spectral imaging may detect skin sensitivity and redness by utilizing the light absorption properties of hemoglobin. This method does not involve fluorescence; instead, it relies on the differential reflectance of light from the skin to visualize and quantify blood flow and inflammation. Hemoglobin, the protein in red blood cells, plays a crucial role in this method. Hemoglobin absorbs light in specific parts of the spectrum, particularly in the visible red (600-700 nm) and green (500-600 nm) ranges. This absorption reduces the amount of light that is reflected back from areas with a high concentration of blood, such as inflamed or red areas of the skin. Multi-spectral imaging detects these changes in light reflectance, allowing it to visualize and quantify the areas of increased blood flow and vascular changes associated with redness and skin sensitivity.

In some embodiments, the image acquisition module 101 may capture images for the analysis module 102 to perform 3D reconstruction of the area of interest. The 3D reconstruction of the area of interest may include the face, neck, abdomen, legs, and arms. Several approaches can be utilized for 3D reconstruction, including but not limited to stereo vision, structured light, and photogrammetry. Stereo vision relies on two or more cameras positioned at different angles to capture images of the same area of interest. By analyzing the disparity between these images, depth information can be determined, allowing for the creation of a 3D model. A typical hardware setup for stereo vision consists of two synchronized cameras mounted on a stable rig, often accompanied by a calibration tool to ensure accurate alignment. Structured light involves projecting a known pattern of light onto the area of interest and capturing the deformation of this pattern with a camera. The distortion of the pattern provides depth information, enabling the construction of a 3D surface model. An example hardware configuration includes a projector to emit the light pattern and a high-resolution camera to capture the reflected light, with both devices calibrated to work in unison. Photogrammetry is another approach that uses multiple overlapping photographs taken from various angles to generate 3D models. The analysis module 102 analyzes these images to identify common points, creating a depth map and ultimately a 3D representation. An example hardware configuration for photogrammetry includes multiple synchronized cameras to capture an area of interest from different angles. As would be understood by one of ordinary skill in the art, other 3D reconstruction approaches may be implemented, without departing from the spirit or scope of the principles of the present disclosure. For example, laser scanning, may be employed, where a laser beam scans the object and measures the time taken for the reflection to return, providing precise depth measurements.

In some embodiments, the image acquisition module 101 may be equipped with optical polarizers. The polarizers may be configured to enable parallel polarization or cross polarization. Parallel polarization involves aligning one polarizer on the light source and another polarizer on the camera in the same orientation. This configuration y captures light that reflects off the surface of the skin, making it ideal for examining superficial features such as texture and fine lines and wrinkles. Parallel polarized light maintains polarization after reflecting off smooth surfaces, thus highlighting surface details while minimizing the visibility of subsurface structures. Cross polarization, on the other hand, places the polarizers on the light source and the camera perpendicular to each other. This configuration blocks the specular (surface) reflection because the reflected light from the surface retains the polarization of the incident light and is filtered out by the perpendicular polarizer on the camera. As a result, cross polarization allows for the capture of light scattered from regions deeper within the skin, providing detailed images of subsurface features such as pigmentation, vascular structures, and erythema. This method is particularly useful for visualizing deeper skin layers, revealing conditions like rosacea, subcutaneous pigmentation changes, and inflammatory responses. The polarizers may also be configured to enable variable degrees of polarization, which involves aligning the polarizers on the light source and the camera at a variable and/or tunable angle.

In some embodiments, the image acquisition module 101 may be physically installed or located within the treatment device 100. In other embodiments, the image acquisition module 101 may be located outside of the treatment device 100. For example, the image acquisition module may be a camera of a mobile device such as a smart phone. In this case, the treatment device 100 and the camera may be connected through wired or wireless communication interfaces. In other embodiments, the image acquisition module 101 may be other off-the-shelf standalone devices that may be connected to the treatment device 100 through wired or wireless communication interfaces.

The analysis module 102 processes and analyzes the images received from the image acquisition module 101. To overcome the shortcomings associated with the state of the art energy-based aesthetic treatment devices, the analysis module 102 processes and analyzes the incoming images to determine meaningful information relevant to treatment planning, monitoring, and/or control. Based on the analysis results, the analysis module 102 may command the treatment module 103 to customize the treatment settings. For example, the analysis module 102 may provide instructions to the treatment module 103 that indicate which treatment setting(s) to adjust and/or a degree to which to adjust the treatment setting(s).

In some embodiments, the analysis module 102 may be implemented using one or more processors built into the treatment device 100. This configuration allows for faster response times and offline operation without the need for external communication links. The one or more processors may be implemented in hardware, firmware, and/or a combination of hardware and software. The one or more processors may include a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), and/or another type of processing component.

In other embodiments, the analysis module 102 may be located external to the treatment device 100. For example, the analysis module 102 may be implemented within a smartphone or tablet connected to the treatment device 100, thereby utilizing the mobile device's processing power, graphical interface, and wireless connectivity. This setup offers a balance between portability and enhanced processing capabilities. In yet other embodiments, the analysis module 102 may reside on a remote server or cloud infrastructure, enabling the use of more advanced ML based algorithms and high-performance computing resources. Cloud-based processing may be especially beneficial for performing computationally intensive tasks such as 3D reconstruction, real-time inference using deep neural networks, or longitudinal tracking of user data. The choice between internal and external placement of the analysis module 102 may be made based on considerations such as desired response time, device size and cost constraints, processing complexity, and network availability.

The analysis module 102 may also include a memory, a storage component, an input component, an output component, and/or a communication interface. A bus may be implemented to permit communication among these components and the one or more processors. The memory may include a random-access memory (RAM), a read only memory (ROM), and/or another type of dynamic or static storage device (e.g., a flash memory, a magnetic memory, and/or an optical memory) that stores information and/or instructions for use by the one or more processors.

The storage component may store information and/or software related to the operation and use of the treatment device 100. For example, the storage component may include a hard disk (e.g., a magnetic disk, an optical disk, a magneto-optic disk, and/or a solid-state disk), a compact disc (CD), a digital versatile disc (DVD), a floppy disk, a cartridge, a magnetic tape, and/or another type of non-transitory computer-readable medium, along with a corresponding drive.

The input component may include a component that permits treatment device 100 to receive information, such as via user input (e.g., a touch screen display, a keyboard, a keypad, a mouse, a button, a switch, and/or a microphone). Additionally, or alternatively, the input component may include a sensor for sensing information (e.g., a global positioning system (GPS) component, an accelerometer, a gyroscope, and/or an actuator). The output component may include a component that provides output information from device 300 (e.g., a display, a speaker, and/or one or more light-emitting diodes (LEDs)).

The communication interface may include a transceiver-like component (e.g., a transceiver and/or a separate receiver and transmitter) that enables treatment device 100 to communicate with other devices, such as via a wired connection, a wireless connection, or a combination of wired and wireless connections. The communication interface may permit treatment device 100 to receive information from another device and/or provide information to another device. For example, the communication interface may include an Ethernet interface, an optical interface, a coaxial interface, an infrared interface, a radio frequency (RF) interface, a universal serial bus (USB) interface, a Wi-Fi interface, a cellular network interface, or the like. One or more of these components may be provided as part of one or more of the other modules described herein.

One function of the analysis module 102 is to monitor the current treatment sub-area. Take a facial and neck treatment for example. In this example, the treatment sub-areas may include forehead, periorbital, eye, undereye, nose, cheek, lips, chin, submental and lateral neck areas. To accomplish this monitoring function, in some embodiments, the analysis module 102 may use machine learning (ML) based algorithms including but not limited to deep neural networks (DNNs), convolutional neural networks (CNNs), support vector machines (SVMs), decision trees and random forests, K-nearest neighbors (KNN). In some embodiments, the analysis module 102 may use image processing algorithms such as those using computer vision. In some embodiments, the analysis module 102 may use both ML based algorithms and image processing algorithms. These ML based algorithms and image processing algorithms can be suitable for making predictions on the class of the current treatment sub-area, e.g., forehead, periorbital, eye, undereye, nose, cheek, lips, chin, submental and lateral neck, etc.

Another function of the analysis module 102 is to monitor the treatment quality. The energy-based aesthetic treatment devices typically have tissue-contacting part(s) of the device (or tissue-contacting part(s)) that deliver(s) the respective energy forms to the tissue to achieve aesthetic effect. Treatment quality requirements in the present invention refer to conditions that are required to deliver safe, comfortable, and effective energy-based aesthetic device treatments. Meeting the treatment quality requirements typically involves the correct placement of the tissue-contacting part of the device with respect to the tissue. The tissue-contacting part may take the form of electrodes for radiofrequency and Electrical Muscle Stimulation (EMS) devices, transducers for ultrasound devices, and transparent lenses for light-based devices. Ensuring correct placement of the tissue-contacting part is generally required for good treatment quality. For example, partial contact of electrodes may result in local concentration of current which may lead to local hot spot, inadequate coupling of ultrasound transducers may result in acoustic energy reflection, and inadequate contact of transparent lenses may result in excessive light reflection and scattering.

To accomplish this function, in some embodiments, the analysis module 102 may use ML based algorithms including but not limited to deep neural networks (DNNs), convolutional neural networks (CNNs), support vector machines (SVMs), decision trees and random forests, K-nearest neighbors (KNN), and/or the like. In some embodiments, the analysis module 102 may use image processing algorithms. In some embodiments, the analysis module 102 may also use both ML based algorithms and image processing algorithms. These ML based algorithms and image processing algorithms can be suitable for making predictions on whether the treatment quality requirements are met. For example, the output of the machine learning algorithm may be a binary classification of whether an electrode is making sufficient contact with the target tissue, whether the transducer is adequately coupled to the target tissue, whether the transparent lens is correctly placed on the target tissue, and so forth. For combination energy-based devices (e.g., one device configured to deliver more than one form of energy), analysis module 102 may use one suitable machine learning algorithm to perform multiclass classification, e.g., outputting data indicating whether the treatment quality requirements for each of the energy modalities are met at the same time. In some embodiments, analysis module 102 may use more than one ML based algorithm, with each algorithm performing binary classification independently or in parallel to determine whether treatment quality requirements for each of the energy modalities are met. Then, the analysis module 102 may combine the classification results of the more than one suitable ML based algorithms as a final classification output.

Yet another function of the analysis module 102 is to monitor whether tissue reactions to the energy-based aesthetic treatments are within a normal range. For a safe, comfortable, and effective treatment, it is important to ensure tissue reactions during the treatment are within normal range. For example, an RF intensity level that is tolerated by normal skin may not be tolerated by sensitive skin. This may cause the sensitive skin to develop excessive skin redness during the treatment, which, if undetected, then as treatment continues, the risk of post treatment adverse effects would be significantly increased. Another example is for ablative skin laser treatments. In this example, the primary endpoint may be characterized with a specific color and/or size of a lesion. Close monitoring of these primary endpoints' visual indicators is critical to ensure the treatment stays within a safe boundary.

To accomplish the function of monitoring tissue reactions, in some embodiments, the analysis module 102 may use ML based algorithms including but not limited to deep neural networks (DNNs), convolutional neural networks (CNNs), support vector machines (SVMs), decision trees and random forests, K-nearest neighbors (KNN). In some embodiments, the analysis module 102 may use image processing algorithms. In some embodiments, the analysis module 102 may also use both ML based algorithms and image processing algorithms. These ML based algorithms and image processing algorithms can be suitable for making predictions on whether the tissue reaction is within normal range. For combination energy-based devices (e.g., one device that delivers one or more forms of energy), analysis module 102 may use an ML based algorithm to perform multiclass classification, e.g., outputting whether the tissue reaction for each of the energy modalities is within normal range at the same moment in time. In some embodiments, the analysis module 102 may use more than one suitable ML based algorithm, with each of the algorithms performing binary classification independently or in parallel to determine whether tissue reaction for each of the energy modalities is within normal range. Then, analysis module 102 may combine the classification results of the ML based algorithms as its classification output.

In some embodiments, the ML based algorithms (commonly referred as machine learning models or data models trained using machine learning) may include but not limited to MobileNet, SqueezeNet, ShuffleNet, EfficientNet, ESPNet, Xcepion, Inception V3, Resnet, DenseNet, VGG, YOLO, SSD, Faster R-CNN, NASNet, PNASNet, UNet, SAM, MnasNet, AmoebaNet, HRNet, RegNet and their variants. The machine learning models may be trained to output key points associated with the treatment area of interest as well as key points associated with the energy based treatment devices. In some embodiments, the machine learning models may output key points of a face that is undergoing (or is soon to undergo) facial treatment. The machine learning models may also output key points associated with the treatment device 100. These key points may be generated at a predetermined time interval or generated in real time while the treatment is being administered.

The current treatment sub-area, the relative speed of the tissue-contacting part of the device with respect to the tissue being treated, the contact quality, the real time tissue reaction to treatment, and/or a dynamic treatment area may be assessed by the analysis module 102 by analyzing the spatial and time relationship between the face key points and the device key points. For example, if the key points associated with the tissue-contacting part of the device fall within the key points that define the cheek area, the current treatment sub-aera can be inferred as cheek. The relative speed of the tissue-contacting part of the device with respect to the tissue being treated may be calculated by dividing the change in distance between the key points associated with the tissue-contacting part of the device and the key points associated with the tissue being treated, by the time during which such change in distance occurs. The contact quality may be assessed by looking at whether the key points associated with the tissue-contacting part of the device are in close proximity to the key points associated with the tissue being treated. If a gap indicative of poor tissue contact is found between the key points associated with the tissue-contacting part of the device and the key points associated with the tissue, the contact quality requirements may be determined as not met. The dynamic treatment area may be calculated by tracking the area covered by the tissue-contacting part of the device over the tissue being treated over a predefined period of time. The area may be computed by finding the outer most boundary of all trajectories of the key points associated with the tissue-contacting part of the device formed on the tissue being treated. In some embodiments, the dynamic treatment area may be updated by the analysis module 102 in a moving average style. In other embodiments, other ML based algorithms could be employed to assess the current treatment sub-area, the relative speed of the tissue-contacting part of the device with respect to the tissue being treated, the contact quality, the real-time tissue reaction to treatment, and the dynamic treatment area, without departing from the spirit or scope of the principles of the present disclosure. Such outputs of ML based algorithms may include object segmentation, for example, the ML based algorithms may segment the energy based treatment device 101 or part of the energy based treatment device 101, and/or the tissue being treated to evaluate their spatial and temporal relationship. Such outputs of the ML based algorithms may include both object segmentation and key points.

The image processing algorithms may include but not limited to the following: thresholding converts grayscale images into binary images by applying a threshold value and is commonly used for background subtraction and basic object segmentation. Edge detection methods (e.g., Canny, Sobel) may be used to detect image boundaries by identifying regions with rapid intensity changes and can be used for object detection, feature extraction, and segmentation. Contour detection techniques can be used to identify the boundaries of objects in binary images and are applicable in shape analysis and segmentation tasks. Connected component labeling assigns unique labels to connected regions in binary images and is widely used in object detection and segmentation workflows. Watershed algorithms, which treat an image as a topographic surface, are useful for separating overlapping objects and performing complex region-based segmentation. K-means clustering, which may also be classified as an unsupervised machine learning method, partitions an image into k clusters based on color or intensity and is commonly used in color quantization and segmentation. GrabCut is an interactive foreground extraction algorithm used to separate objects from the background. Mean Shift Segmentation, another method that may be considered unsupervised learning, shifts each data point toward the mean of its neighborhood and is frequently used for segmentation and object tracking. Graph Cut Segmentation formulates the segmentation task as a graph-based optimization problem to identify the minimal cut and can be used for image segmentation purposes.

Yet another function of the analysis module 102 is to perform 3D reconstruction of the area of interest. The 3D reconstruction of the area of interest may include but not limited to the face, neck, abdomen, legs, and arms. As disclosed elsewhere herein, stereo vision algorithms such as block matching or semi-global matching may be employed to compute disparity maps from the stereo image pairs. These algorithms assess pixel intensity differences between the two images to estimate depth. Once the disparity is calculated, it is converted into a 3D point cloud using the camera's intrinsic parameters, resulting in a 3D representation of the area of interest. Post-processing steps, including filtering and refinement techniques, may be applied to enhance the quality of the 3D model.

As disclosed elsewhere herein, a structured light approach may be employed which involves projection of a light pattern onto the area of interest while simultaneously capturing images with a camera. The analysis module 102 may analyze the captured images using algorithms like edge detection and phase shifting to identify distortions in the light pattern. This data is then processed to compute a depth map, which is converted into a 3D point cloud. Additionally, calibration algorithms may be employed to ensure that the projector and camera are accurately aligned, minimizing measurement errors. As disclosed elsewhere herein, a photogrammetry approach may be employed which begins with feature extraction algorithms, such as SIFT (Scale-Invariant Feature Transform) or SURF (Speeded-Up Robust Features), to identify key points across multiple images. These points may be matched to determine correspondences, and bundle adjustment algorithms may be used to optimize the camera parameters and 3D point locations simultaneously. Once the 3D point cloud is generated, meshing algorithms, such as Delaunay triangulation, may be used to create a continuous surface representation. Texture mapping algorithms may be used to apply surface textures derived from the original photographs, enhancing the realism of the 3D model.

In some embodiments, ML based algorithms may be employed to perform 3D reconstruction of the area of interest including but not limited to the face, neck, abdomen, legs, and arms using monocular RGB images. This approach may serve as an alternative or complement to traditional stereo vision, structured light, or photogrammetry techniques, especially when hardware constraints limit multi-view capture. In such embodiments, the image acquisition module 101 may acquire a single high-resolution RGB image of the area of interest, which is then processed by the analysis module 102 using depth estimation models. One exemplary model is Depth Anything, a transformer-based architecture trained on a large and diverse dataset to produce dense depth maps from monocular input images. Other models that may be used include MiDaS (Mixed Depth for All Scales), DPT (Dense Prediction Transformer), Boosting Monocular Depth (a fusion-based approach that refines initial depth predictions), and NeRF-based neural rendering systems, each offering unique trade-offs in terms of accuracy, generalization, and computational efficiency. Once a depth map is predicted from the monocular image, the analysis module 102 may use the camera's intrinsic parameters—such as focal length and optical center—to convert each pixel into a 3D point in camera space. One method to convert each pixel into a 3D point in camera space may involve using pinhole projection equations:

$$X = ( u - {c}_{x} ) Z / {f}_{x} , Y = ( v - {c}_{y} ) Z / {f}_{y} , Z = depth ( u , v )$$

where (u, v) are pixel coordinates and Z is the estimated depth. The result is a dense 3D point cloud representation of the area of interest. These ML based 3D reconstruction techniques allow for lightweight yet powerful modeling of the area of interest and are particularly advantageous in scenarios where only a single camera is available or where minimal setup time is desired.

Yet another function of analysis module 102 is to calculate volume and/or volume changes based on 3D models of the area of interest reconstructed at different time points. These different time points may include pre-treatment phase and the treatment phase. Pre-treatment data may be used for personalizing the treatment plan while the treatment data may be used to monitor treatment related indicators. To achieve this, several algorithms may be employed by analysis module 102, each leveraging advanced computational techniques to assess volumetric differences effectively. One approach is the use of 3D mesh comparison algorithms, which allow for precise measurement of changes in the geometry of the reconstructed models. Techniques such as iterative closest point (ICP) or shape deformation analysis can align two meshes captured at different time points. The ICP algorithm iteratively refines the alignment of the point clouds or meshes by minimizing the distance between corresponding points, providing a robust framework for assessing changes in volume. Once aligned, volume calculation can be executed through several methods, including convex hull algorithms and triangulation-based volume computations. The convex hull method computes the smallest convex shape that encompasses the entire 3D model. By comparing the convex hulls of the models at different time points, the algorithm can derive volume changes by subtracting the volumes of the two hulls. This method is particularly useful for irregularly shaped objects, such as the human face or abdomen, as it captures the overall volume without being overly sensitive to surface noise. Another method for calculating volume changes is through voxel-based analysis. In this approach, the 3D models are converted into a voxel representation, where the space is divided into a grid of small cubes (voxels). By using binary occupancy grids, where each voxel is classified as occupied or unoccupied based on the presence of the reconstructed surface, the volume can be calculated directly from the number of occupied voxels. Algorithms can also analyze changes in voxel occupancy over time, allowing for the calculation of gains or losses in volume. Advanced techniques such as Marching Cubes can also be employed to extract a polygonal mesh from the voxel data, further aiding in visualizing and analyzing volumetric changes. In addition to geometric approaches, ML based algorithms may be applied to assess volume changes in specific regions of interest. For instance, convolutional neural networks (CNNs) can be trained to identify and quantify changes in volume based on segmented regions of the 3D models. Without wishing to be bound by theory, other algorithms that calculate volume and volume changes, as commonly understood by one of ordinary skill in the art to which the invention pertains, could be used in analysis module 102 without departing from the spirit or scope of the principles of the present disclosure.

In some embodiments, the treatment module 103 may generate and deliver energy to the intended tissue (e.g., tissue in a current treatment sub-area as determined based on the location of the tissue-contacting part of the treatment device 100). The energy forms may include but not limited to radiofrequency (RF), ultrasound, intense pulsed light (IPL), laser, low intensity light, other energy forms described herein, and/or the like. The treatment module 103 performs the treatment in real-time, based on the commands from the analysis module 102 in response to the image data captured by the image acquisition module 101.

In some embodiments, the treatment module 103 may include an energy emission component configured to deliver energy as part of an energy-based aesthetic treatment. The energy emission component may include one or more hardware sub-components configured to emit energy for delivery to tissue during aesthetic treatment procedures. For example, the energy emission component may include one or more energy transducers, an energy coupling interface, a power control and distribution sub-system, a thermal and safety management sub-system, a mechanical integration and motion system, and/or the like. The energy transducers may include RF electrodes, laser emitters, IPL lamps, ultrasound transducers, resistive heaters or thermoelectric elements, cryogenic modules, piezoelectric or vibration actuators, and/or the like. The energy coupling interface may include a tissue-contacting surface, an acoustic coupling media reservoir, optical waveguides or lenses, motion or pressure sensors, and/or the like. The power control and distribution subsystem may include energy control circuits, multiplexing circuits, real-time feedback modules, and/or the like. The thermal safety and management subsystem may include thermal sensors, over-temperature cutoff circuitry, ground fault or impedance monitoring subsystems, and/or the like. The mechanical integration and motion system may include motors or actuators (in automated embodiments) to move the applicator across the tissue, encoders or positional tracking systems to support determining a dynamic treatment area and relative speed computation, and/or the like.

The energy emission assembly may include, or may be integrated within, a treatment applicator, which is configured to deliver one or more types of energy to a treatment area. The treatment applicator may include or be operatively coupled to one or more of the example sub-components described above.

The user interface (UI) module 104 may be configured to provide information for display via a user interface accessible to the operator of the treatment device 100. The UI module 104 may include touchscreen, microphone, and speaker to enable text, voice, visual information exchange, and/or the like. The UI module 104 may be located inside or outside of the treatment device 100.

In some embodiments, the UI module 104 may display a personalized treatment plan to the operator. For example, the UI module 104 may be used to communicate the personalized treatment plan generated by the analysis module 102 prior to the commencement of the treatment. This plan may include information such as recommended treatment intensity settings (or output power levels) for different treatment sub-areas, treatment duration for each sub-area, treatment sequence of the sub-areas, and any user-specific adjustments (for example, user-specific risk areas that have contraindicated skin conditions). This plan may also include user-specific treatment regimen, an exemplary regimen may be performing the treatment 5 times a week for a duration of 4 weeks. In certain embodiments, the UI module 104 may also include visual overlays or facial mapping, allowing the user to see a region-by-region breakdown of the intended treatment strategy. This enables the user to understand the rationale behind the personalization and facilitates more confident and informed use of the device.

In some embodiments, the UI module 104 may display real-time treatment feedback and warnings to the operator, and to receive information/commands from the operator. The UI module 104 may provide real-time feedback during ongoing treatment. This real-time visualization may include the current treatment sub-area, the dynamic treatment area, the contact quality indicators, the tissue reaction, the relative speed of the tissue-contacting part of the device with respect to the tissue being treated. By presenting this information as it is generated in a real-time manner, the UI module 104 enables the operator to take timely corrective actions, thereby improving both treatment safety and effectiveness. In home-use scenarios, real-time feedback can also function as a guided user experience, enhancing safety by alerting the user to prohibited actions, including but not limited to applying treatment too close to the eyes, or continuing treatment after abnormal skin reactions are detected.

In some embodiments, the UI module 104 may be physically integrated with the treatment device 100 itself, e.g., via a built-in touchscreen or display, or it may be implemented externally. For example, in scenarios where device size, cost, or user convenience are critical, the UI module 104 may be realized as a software application on a smartphone, tablet, or other connected computing device. In such embodiments, the treatment device 100 may communicate with the external UI module wirelessly (e.g., via Bluetooth, Wi-Fi) or through a wired connection. This flexible architecture allows for broader accessibility and leverages the computational and display capabilities of modern mobile devices to enhance the user experience.

Some embodiments described herein involve an example process of customizing an energy-based aesthetic treatment. Example embodiments for performing the steps of the process for customizing the treatment are described below. However, additional example details and/or embodiments, such as those describing how specific steps of the method may be implemented, are described elsewhere herein.

In some embodiments, the example process includes obtaining obtain image data identifying a treatment area. For example, one or more cameras of the image acquisition module 101 may be positioned to capture image data of the treatment area. If the treatment has already started at the time of image capturing, the one or more cameras of the image acquisition module 101 may further capture image data identifying a tissue-contacting part of the treatment device 100. The image acquisition module 101 may then provide the image data to the analysis module 102.

When performing an energy-based aesthetic treatment, the treatment area contains sub-areas with different anatomical characteristics. These differing anatomical characteristics can influence the ideal treatment settings to apply when delivering energy to a given treatment sub-area. For example, when delivering RF energy to a face, different parts of the face have different skin thicknesses. As such, an optimal treatment intensity level changes based on the thickness of the skin. Notably, if the energy modality is something other than RF energy (e.g., ultrasound energy, electrical pulses, light-based energy, etc.), then a different treatment setting needs to be adjusted to optimize the energy delivery. For example, if the energy modality is ultrasound energy, a depth of a bone (relative to a skin surface) in a current treatment sub-area may influence the optimal focal depth level needed to optimize the energy delivery.

To address this, in some embodiments, the example process includes selecting an anatomical classification criterion based on a type of energy modality used for the energy-based aesthetic treatment. For example, the treatment device 100 (e.g., using the analysis module 102) may select an anatomical classification criterion based on a type of energy modality used for the energy-based aesthetic treatment.

The type of energy modality may be RF energy, ultrasound energy, electrical pulses, light-based energy, and/or one or more other forms of energy delivery described herein. The anatomical classification criterion refers to an anatomical characteristic with a measurable or observable property that differs across regions of tissue in the treatment area. The anatomical classification criterion may include a skin thickness, a depth of a bone relative to a skin surface, a depth of a muscle or a nerve fiber relative to the skin surface, an orientation of the muscle or the nerve fiber, a skin condition, and/or the like. Specific anatomical characteristics each have a measurable or observable property that differs across regions of tissue in the treatment area. Specific examples are provided further herein. However, to provide one example, a skin thickness may be an anatomical classification criterion and different regions of tissue in the treatment area may have observable properties such as being thick or thin. By selecting the anatomical classification criteria, the treatment device 100 is able to determine whether tissue being treated is thick or thin and can adjust treatment settings to optimize the energy being delivered during the treatment.

In some embodiments, the example process includes determining, by analyzing the image data using the anatomical classification criterion, a plurality of treatment sub-areas within the treatment sub-area. For example, the treatment device 100 (e.g., using analysis module 102) may determine, by analyzing the image data using the anatomical classification criterion, a plurality of treatment sub-areas within the treatment sub-area. As noted above, it is important to determine the treatment sub-areas because anatomical characteristics of tissue within the total treatment area can vary greatly from one treatment sub-area to another.

Moreover, different treatment sub-areas are present for different anatomical characteristics. For example, a set of treatment sub-areas may be determined based on regions of tissue in a face having thick or thin skin, whereas a different set of treatment sub-areas would need to be determined based on regions of tissue in the face having different bone density or bone depth relative to a skin surface. To provide several other examples: different treatment sub-areas may have tissues containing different skin thicknesses and different levels of skin hydration, which may contribute to varying physical properties such as electrical impedance, acoustic impedance and optical transmittance; different treatment sub-areas may have tissues containing nociceptors that exhibit different activation thresholds for pain stimuli (for instance, activation thresholds for heat induced pain); different treatment sub-areas may exhibit distinct distributions of muscle fibers and nerve endings, including differences in type, depth, density, and anatomical orientation. Additionally, different treatment sub-areas may present varying underlying anatomical structures. For example, the cheek region may contain a thick layer of subcutaneous fat, while the forehead and periorbital regions may have relatively thin, soft tissue overlying shallow bone. These anatomical differences across the treatment area highlight the need for sub-area-specific customization of treatment settings.

As described above, to determine the plurality of treatment sub-areas, the treatment device 100 (e.g., using the analysis module 102) may analyze the image data using the anatomical classification criterion. In some embodiments, the image data may be analyzed using one or more of the ML based algorithms or image processing algorithms described elsewhere herein. In some embodiments, the analysis may be driven by known anatomical characteristics of the human body. For example, in the human face, it is known that certain regions of tissue have thicker skin while other regions of tissue have thinner skin. As such, in some embodiments, the treatment device 100 may use an ML based algorithm or an image processing algorithm to determine key points that define treatment sub-areas based on the selected anatomical classification criterion. In this way, the treatment device 100 determines treatment sub-areas that contain, or correspond to, regions of tissue that have an anatomical characteristic with a measurable or observable property that is capable of influencing the energy delivery of the treatment.

In some embodiments, while the energy-based aesthetic treatment is being performed, the example process includes determining a current treatment sub-area, of the treatment area, that is actively being treated or targeted for treatment. For example, the treatment device 100 (e.g., using analysis module 102) may determine a current treatment sub-area, of the treatment area, that is actively being treated or targeted for treatment. The treatment device (e.g., using analysis module 102) may determine the current treatment sub-area based on a position of the tissue-contacting part of the treatment device 100 relative to one of the plurality of treatment sub-areas. For example, in some embodiments, the image acquisition module 101 may include visible light digital camera(s), such as a charge-coupled device (CCD) or a complementary metal-oxide semiconductor (CMOS) camera, which may be configured to capture both the tissue-contacting part of the treatment device 100 and the treatment sub-area. The captured images are transmitted to the analysis module 102 for further analysis. As the treatment is being administered, the operator will typically treat specific areas one at a time. For example, a facial treatment may involve treating a forehead region, right and left periorbital regions, and right and left cheek regions. When the operator intends to deliver energy to a specific sub-area (e.g., the forehead), the tissue-contacting part of the treatment device 100 will be located in the intended treatment sub-area (e.g., the forehead). As such, the treatment device (e.g., using analysis module 102) may analyze the image data that contains both the intended treatment sub-area and the tissue-contacting part of the treatment device 100 and can determine the current treatment sub-area based on the relative positioning of the tissue-contacting part of the treatment device 100.

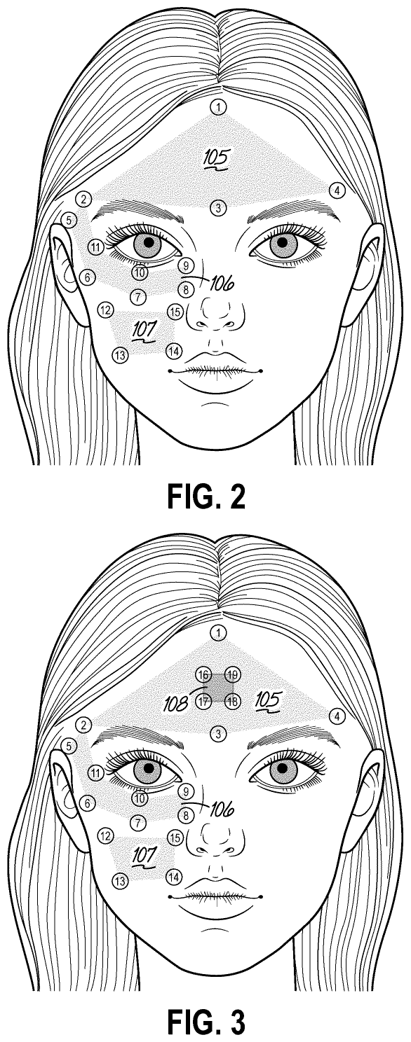

FIG. 2 shows an example key point distribution for a facial treatment: key points 1, 2, 3, and 4 delineate a treatment sub-area 105 for the forehead; key points 2, 5, 6, 7, 8, 9, 10, and 11 delineate a treatment sub-area 106 for the right periorbital region; key points 12, 13, 14, and 15 delineate a treatment sub-area 107 for the right cheek area. While not shown, treatment sub-areas may also include a treatment sub-area for the left periorbital region and a treatment sub-area for the left cheek area. It is to be understood that this is provided by way of example, and that in practice, a treatment area may include any number of treatment sub-areas, and any number of key points defining boundaries of respective treatment sub-areas.

In some embodiments, a data model trained using machine learning may be trained to output key points associated with the tissue-contacting part of the treatment device 100. For example, the data model may be trained to simultaneously output the key points associated with the treatment sub-areas and the key points associated with the tissue-contacting part of the treatment device 100. As shown in Error! Reference source not found., and by reference number 108, the tissue-contacting part of the treatment device 100 is denoted by a square, where boundaries of the square are delineated using key points 16, 17, 18, and 19. By analyzing the spatial relationship between the key points of treatment sub-area 105 and key points of the contacting-part of the treatment device 100, the analysis module 102 may determine the current treatment sub-area. Using FIG. 3 as an example, the analysis module 102 may determine that the current treatment sub-area is the forehead, which is delineated by key points 1, 2, 3, and 4.

In some embodiments, a data model using machine learning may be trained to directly output the current treatment sub-area, without relying on the key point-based methods disclosed above. In these embodiments, the training dataset may include labeled images corresponding to specific treatment sub-areas, such as “forehead,” “right periorbital,” “right cheek,”, and other relevant classifications such as “in contact” and “not in contact”. In this way, determining the current treatment sub-area can be characterized as a multiclass classification problem, where the data model outputs a single class label that represents the current treatment sub-area based on the input image. The advantage of this approach lies in its computational efficiency and simplified inference pipeline. For example, post-processing of key points is not required as the data model generates a direct prediction of the current treatment sub-area. This may be particularly beneficial in resource-constrained environments or for real-time applications where rapid decision-making is desirable.

The machine-learning driven embodiments described above are provided by way of example. In practice, other types of machine learning techniques may be implemented. For example, the data model may be trained to implement a semantic segmentation approach. In this case, the tissue-contacting part of the treatment device 100 and the treatment sub-areas may be individually segmented within the captured image. The analysis module 102 may then determine the current treatment sub-area by analyzing the spatial relationship between the segmented region corresponding to the tissue-contacting part of the device 100 and the segmented treatment sub-areas. This segmentation-based approach may be particularly advantageous when finer spatial resolution is desired or when the key point-based or classification-based methods lack sufficient granularity for accurate localization.

In some situations, an operator of the treatment device 100 may follow a treatment plan whereby energy is to be delivered to specific treatment sub-areas within the treatment area. Referring to the example described above, the treatment area may have five treatment sub-areas and the operator may delivery energy to the first treatment sub-area, the second treatment sub-area, . . . , and to the fifth treatment sub-area. In some situations, while performing the treatment, the operator may mistakenly or unknowingly treat tissue within a risk area. A risk area, as used herein, refers to a region of tissue that presents potential safety or efficacy concerns during energy-based aesthetic treatment. Such regions may include, but are not limited to: inflamed areas characterized by visual redness, swelling, elevated temperature, or increased vascularization, sensitive areas identified based on anatomical mapping, prior procedures, or visual indicators of skin fragility or high nociceptor density, sunken areas identified through 3D imaging as regions exhibiting abnormal volume loss or negative contour deviation, and/or the like.

Tissue inflammation, tissue sensitivity, and/or tissue characterized as sunken may be detectable by image analysis techniques described herein. For example, inflammation may be detectable due to increased skin redness (e.g., through color analysis of RGB or multispectral images), elevated local temperature (e.g., measurable via IR thermography or contact-based temperature sensors), swelling or edema (e.g., detectable via 3D facial reconstruction or image based depth estimation), increased vascularization or blood flow (e.g., which can be inferred using multispectral imaging or near-infrared analysis targeting hemoglobin absorption bands), texture irregularities or shine (e.g., detectable from high resolution optical imaging), presence of lesions, pustules, or nodules (e.g., detectable by ML based object detection or segmentation), and/or the like. While inflammation may be detected using a data-driven analysis, location-based rules (e.g., common areas of irritation) may also serve as default risk area indicators in situations where real-time data is unavailable.

To provide another example, sensitivity may be detected from high nociceptor density areas (e.g., lips, periocular area, etc., based on anatomical mapping), low pain threshold regions (e.g., which may be inferred through known dermatome or nerve distribution maps), recent procedural sites or damaged skin (e.g., detected through visual cues or user input), dry, cracked, or thin epidermis (e.g., measurable via skin hydration sensors or image based skin quality analysis), presence of atopic conditions such as eczema or psoriasis (e.g., detected using trained ML classifiers applied to the image data), and/or the like. To provide another example, a sunken area, which refers to a localized facial depression (which can, for example, result from fat loss or volume depletion), may be detectable from negative surface contain deviations (e.g., detected through 3-D facial scans, depth mapping, or stereo vision), volume loss relative to a reference facial geometry (e.g., calculated using volume differential between a patient-specific 3D model and a matched average template), depression area and depression volume (e.g., measured via mesh registration techniques or geometric analysis), and/or the like. Embodiments of the present invention may distinguish between clinically contraindicated sunken areas (e.g., which may result in immediate treatment settings adjustments) from mild sunken regions (e.g., which may result in simple pre-cautionary actions being taken).

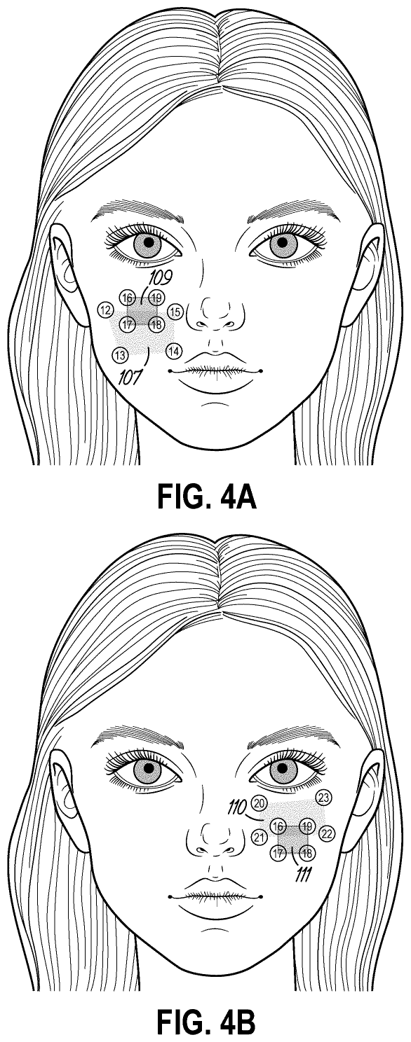

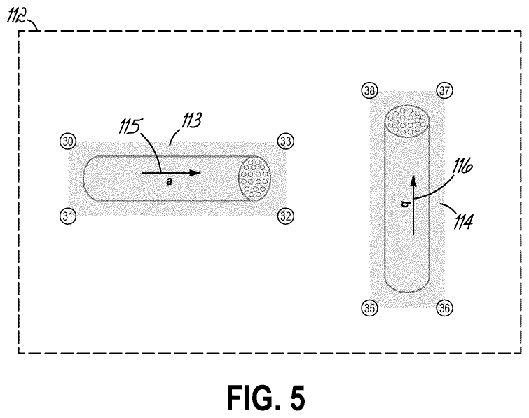

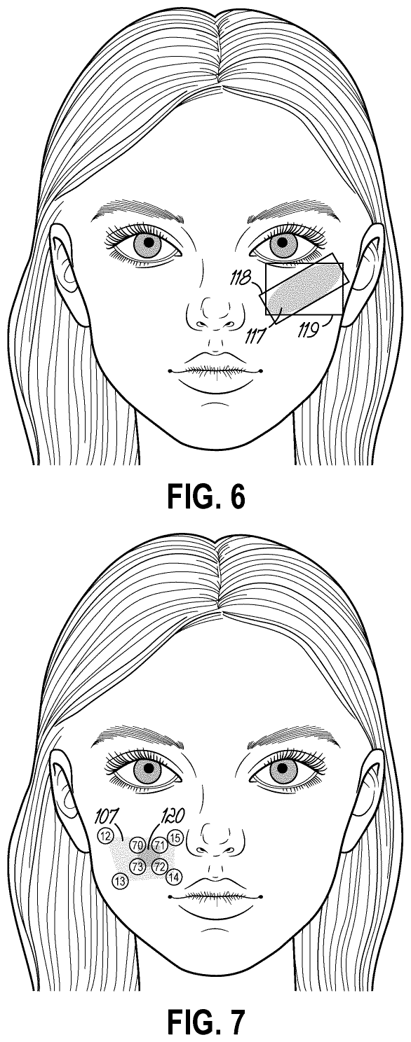

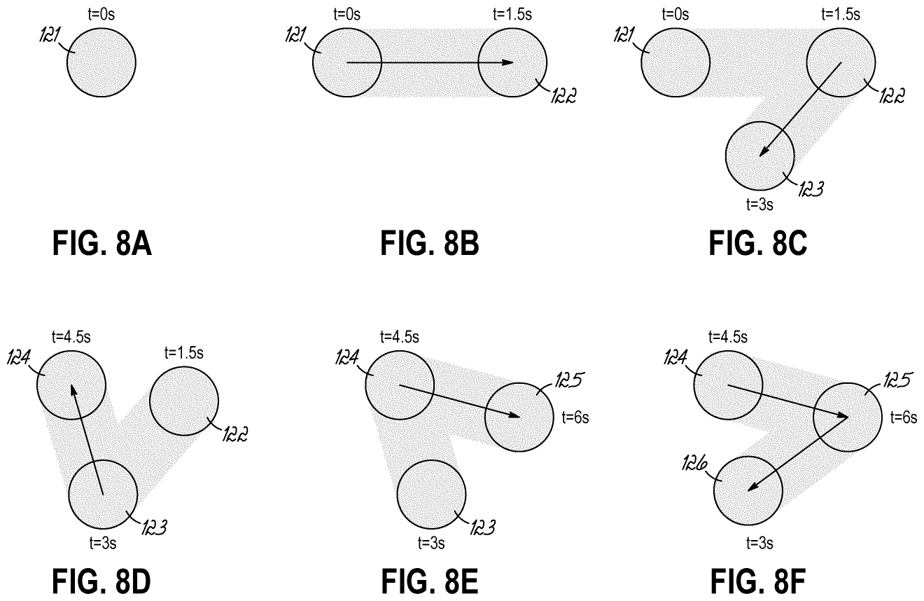

To prevent these situations from occurring, or to allow the operator to quickly correct course, in some embodiments, the treatment device 100 (e.g., using analysis module 102) may determine that the operator is treating tissue within a region of tissue identified as a risk area. For example, the treatment device 100 may determine that the tissue-contacting part of the treatment device 100 at least partially overlaps with a risk area representing a region of tissue that is inflamed, sensitive, sunken, and/or the like. In FIG. 4B, the tissue-contacting part of the treatment device 100, as delineated by key points 16, 17, 18, and 19, overlaps a risk area 110 delineated by key points 20, 21, 22, and 23. In this case, the analysis module 102 may determine that the operator is treating within the risk area 110.

A risk area may not be categorically excluded from treatment; rather, the risk area may be assessed using one or more quantitative metrics (sometimes referred to as risk scores), such as an inflammation score, a sensitivity score, a depression score, and/or the like. These quantitative metrics or risk scores may be used as a way of measuring the risk associated with treating tissue included in the risk area. In some embodiments, for the scenario involving the risk area, the treatment device 100 (e.g., using analysis module 102) may determine a risk score to assess a risk of treating tissue included in the risk area. The treatment device 100 may then compare the risk score to a threshold risk score. When the determined risk score is below, but near, the threshold risk score, the treatment device 100 may generate and provide for display via the user interface module 104, a warning or message alerting the operator to proceed with caution. When the determined risk score exceeds the threshold risk score, the analysis module 102 may adjust one or more treatment settings and/or may generate and provide a warning or recommendation for display via the user interface module 104. Adjusting one or more treatment settings may, for example, include interrupting the ongoing treatment. Such interruption may include turning off the device, pausing the treatment, or reducing the treatment intensity (e.g., lowering the output power of the treatment device 100), and/or the like.

In other situations, while performing the treatment, the operator may mistakenly treat tissue outside of an intended treatment area (e.g., a sub-area in which the operator is intended to treat). In these situations, the treatment device 100 may determine that the operator is treating tissue outside of the intended treatment sub-area. For example, the analysis module 102 (e.g., using analysis module 102) may use one or more of the ML based algorithms and/or image processing algorithms described herein to determine whether the operator is treating tissue outside of the intended treatment sub-area. In FIG. 4A, the current treatment sub-area 107 is delineated using key points 12, 13, 14, and 15. The area 109 covered by tissue-contacting part of the treatment device 100 is delineated using key points 16, 17, 18, and 19. As can be seen in FIG. 4A, the tissue-contacting part of the treatment device 100 covers the area 109 which is partially within the current treatment sub-area 107. Based on this spatial relationship, the analysis module 102 may determine that the operator is treating tissue outside of the current treatment sub-area.

In some embodiments, the threshold driven analysis described with respect to the risk area may be implemented when the treatment device 100 determines that the tissue-contacting part of the treatment device 100 is outside of the intended treatment sub-area. For example, the treatment device 100 (e.g., using analysis module 102) may determine a measured severity level to assess a degree to which the operator is treating tissue outside of the current treatment sub-area. The treatment device 100 may then compare the measured severity level to a threshold severity level. When the measured severity level is below the threshold severity level, the treatment device 100 may generate and provide for display via the user interface module 104, a warning or message alerting the operator to proceed with caution. When the measured severity level exceeds the threshold severity level, the analysis module 102 may adjust one or more treatment settings and/or may generate and provide a warning or recommendation for display via the user interface module 104. Adjusting one or more treatment settings may, for example, include interrupting the ongoing treatment. Such interruption may include turning off the device, pausing the treatment, or reducing the treatment intensity (e.g., lowering the output power of the treatment device 100), and/or the like.

As described in the embodiments above, when the analysis module 102 determines that the operator is treating outside the intended treatment sub-area and/or within a risk area, the analysis module 102 may generate a message or a warning to prompt the operator to take appropriate corrective actions. Such message or warning may include a voice prompt, a text prompt displayed via the UI module 104, or another commonly understood alert signal recognizable to one of ordinary skill in the art. In response to these conditions, as described above, the analysis module 102 may also send commands to the treatment module 103 to interrupt the ongoing treatment. In one embodiment, the analysis module 102 may further direct the UI module 104 to display real-time visual indicators highlighting the location of the tissue-contacting part of the treatment device 100 in relation to the designated treatment sub-area and/or any risk area. For example, regions of improper placement may be overlaid with distinct visual markers or color-coded boundaries to indicate deviation from the treatment plan. These visual cues are intended to assist the operator in promptly correcting the device position, thereby ensuring both treatment safety and protocol compliance.

After determining the current treatment sub-area, the analysis module 102 may continue to customize the treatment settings accordingly. In the context of the present invention, the term treatment settings refers to device-specific operational parameters that may vary depending on the type of energy-based aesthetic device in use. These settings may include, but are not limited to, treatment intensity, pulse duration, frequency, energy delivery mode, depth of penetration, turning off the device, pausing the treatment, and/or the like. The specific treatment parameters applicable to each type of energy modality (e.g., RF energy, ultrasound energy, light-based energy, and/or the like), will be described in detail in the various embodiments disclosed herein.

In some embodiments, the treatment module 103 may use radiofrequency (RF) as the energy modality for facial treatment. In such treatments, the dermis absorbs the majority of the RF energy. Therefore, a thicker dermis requires a greater amount of energy to reach the same therapeutic temperature compared to a thinner dermis. To account for this variation, the total facial treatment area may be divided into sub-areas based on skin thickness. For example, as shown in Error! Reference source not found., the treatment sub-area 106, which is delineated by key points 2, 5, 6, 7, 8, 9, 10, and 11, may exhibit thinner skin compared to the treatment sub-area 107 which is delineated by key points 12, 13, 14, and 15. Accordingly, the treatment sub-area 106 may be characterized as a thin area, and the treatment sub-area 107 as a thick area. When the analysis module 102 determines that the current treatment sub-area is a thick area, the analysis module 102 may command the treatment module 103 to increase the treatment intensity, e.g., the RF output power. Conversely, when the current treatment sub-area is identified as a thin area, the analysis module 102 may command the treatment module 103 to decrease the treatment intensity. In these embodiments, treatment customization involves dynamically adjusting the RF output power based on the skin thickness of the current treatment sub-area. It is further noted that the classification of skin thickness is not limited to a binary scheme. In some embodiments, the facial treatment area may be divided into more granular categories, such as very thick, thick, intermediate, thin, and very thin, to enable a higher degree of customization.

In some embodiments, the treatment module 103 may use focused ultrasound as the energy modality for facial treatment. In such treatments, it is essential for safety reasons to avoid focusing ultrasound energy directly onto underlying bone structures. Accordingly, the total facial treatment area may be divided into sub-areas based on the depth of the bone relative to the skin surface. For example, as shown in Error! Reference source not found., the bone beneath the treatment sub-area 105, which is delineated by key points 1, 2, 3, and 4, is known to be shallower than the bone beneath the treatment sub-area 107 which is delineated by key points 12, 13, 14, and 15. The treatment sub-area 105 may therefore be characterized as a bony area, while the treatment sub-area 107 may be characterized as a non-bony area. When the analysis module 102 determines that the current treatment sub-area is a bony area, the analysis module 102 may command the treatment module 103 to decrease the focal depth of the ultrasound energy in order to avoid depositing energy too close to the bone. Conversely, when the current treatment sub-area is identified as a non-bony area, the analysis module 102 may increase the focal depth to better target deeper tissue layers. In these embodiments, customization involves dynamically adjusting the focal depth of the ultrasound energy based on the anatomical classification of the current treatment sub-area—e.g., whether the sub-area is classified as bony or non-bony. It is further noted that the classification need not be limited to a binary distinction. In some embodiments, the analysis module 102 may estimate the actual depth (e.g., in millimeters) between the skin surface and the bone, and use this measurement as an input for determining an optimal focal depth. This continuous or multi-level depth mapping may allow for more precise ultrasound treatment planning.

In some embodiments, the treatment module 103 may deliver electrical pulses for Electrical Muscle Stimulation (EMS) or electrical nerve stimulation. In such treatments, the target muscle or nerve fibers may reside at varying depths beneath the skin surface, depending on the anatomical location. Muscle or nerve fibers located at greater depths may require electrical waveforms with increased amplitude to ensure effective stimulation, whereas those situated at shallower depths may respond adequately to waveforms of lower amplitude. To accommodate this variability, the total treatment area may be divided into treatment sub-areas based on the estimated depth of the target muscle or nerve fibers. For example, the analysis module 102 may be configured to output key points that delineate a treatment sub-area containing deeper-lying muscle or nerve fibers, and to output key points delineating sub-areas where such structures are closer to the skin surface. When the analysis module 102 determines that the current treatment sub-area corresponds to a region of tissue with deep muscle or nerve fibers, the analysis module 102 may command the treatment module 103 to deliver an electrical waveform with increased amplitude. Conversely, if the current treatment sub-area is identified as having shallow muscle or nerve fibers, the analysis module 102 may reduce the amplitude of the waveform accordingly.

It is also believed that the direction of the stimulating current is an important factor in the effectiveness of electrical muscle or nerve stimulation treatments, with current aligned parallel (e.g., longitudinally) to the muscle or nerve fibers generally resulting in stronger contractions and improved treatment outcomes. Therefore, the total treatment area may also be divided into sub-areas based on the orientation of the muscle or nerve fibers. For example, the analysis module 102 may output key points 30, 31, 32, and 33, which delineate a treatment sub-area 113 containing muscle or nerve fibers oriented along a longitudinal direction a, as illustrated in Error! Reference source not found. Similarly, the analysis module 102 may output key points 35, 36, 37, and 38, which delineate a treatment sub-area 114 containing muscle or nerve fibers oriented along a longitudinal direction b, as illustrated in Error! Reference source not found. When the analysis module 102 determines that the current treatment sub-area corresponds to a region with longitudinal direction a, the analysis module 102 may command the treatment module 103 to output an electrical current that is substantially aligned with direction a. Likewise, if the current treatment sub-area is associated with direction b, the analysis module 102 may command the treatment module 103 to align the electrical current accordingly. In this manner, the alignment of the stimulating current is customized to match the anatomical direction of the underlying muscle or nerve fibers, thereby improving stimulation efficiency. In alternative embodiments, when such directional alignment cannot be fully automated—such as in cases involving adhesive electrodes that must be manually positioned by the operator—the analysis module 102 may provide real-time visual or textual guidance via the UI module 104. For example, the UI module 104 may display directional overlays, alignment arrows, or placement instructions to assist the operator in manually aligning the electrodes with the anatomical fiber orientation of the identified treatment sub-area. This functionality allows for increased treatment precision, even in systems where hardware constraints limit full automation.

In some embodiments, treatment customization may involve determining treatment sub-areas based on skin condition(s). Distinct skin conditions may require different treatments to ensure safety, comfort, and efficacy. The skin conditions considered in this context may include, but are not limited to, acne, skin inflammation, pigmentation disorders, tattoos, and other localized dermatological features.

In some embodiments, when performing facial treatment, treatment sub-areas corresponding to different skin conditions may be detected and/or segmented using a data model trained using one or more machine learning techniques. In these embodiments, the training dataset may include pixel-wise labeled ground truth annotations of various skin conditions, enabling the ML-based algorithms to be trained for pixel-wise segmentation of those conditions. Once segmentation is performed, the areas encompassing the segmented pixels—referred to as segmentation masks—may be defined as treatment sub-areas associated with their respective skin conditions (117 in Error! Reference source not found.). These treatment sub-areas may also be represented using bounding boxes. The bounding boxes may take the form of axis-aligned bounding boxes (619 in Error! Reference source not found.) or, more preferably, rotated minimum-area bounding boxes (118 in Error! Reference source not found.). Rotated minimum-area bounding boxes offer a tighter fit around irregular segmentation masks, thereby providing a more accurate and efficient representation of the region of interest. These bounding boxes may be generated either directly using one or more machine learning techniques as part of the model's output or as a result of post-processing applied to the segmentation mask. For example, the training dataset may include bounding boxes that enclose the annotated skin conditions, in which case the ML-based algorithm may be trained to predict bounding boxes directly.

Alternatively, the bounding boxes may be computed after segmentation using a defined post-processing workflow. In one embodiment, a rotated minimum-area bounding box may be generated by: (1) identifying the contour points from the segmentation mask; (2) computing the convex hull—e.g., using the Graham Scan algorithm—to account for potential concave shapes; and (3) finding the smallest-area rectangle that can freely rotate to fit around the convex hull. This method ensures a geometrically compact and orientation-aware representation of segmented skin condition areas, which can then be used to define the treatment sub-areas for further customization.

As discussed elsewhere herein, the image acquisition module 101 may be equipped with fluorescent imaging capabilities to facilitate the determination of treatment sub-areas with different skin conditions by the analysis module 102. For example, to detect porphyrins, which are associated with acne due to their production by Propionibacterium acnes bacteria, near-UV light with an excitation wavelength of 400-410 nm may be used. When exposed to this light, porphyrins fluoresce red, emitting light at wavelengths between 600-650 nm. This red fluorescence highlights areas of bacterial activity, aiding in the identification and treatment of acne. To detect pigmentation, particularly due to melanin, UV light within the 320-400 nm range may be used. Pigmentated areas emit light in a broad spectrum, often around 420-560 nm. This emission allows for the visualization of pigmented areas, facilitating the assessment of pigmentation disorders.

Alternatively, the imaging acquisition module 101 may be equipped with multi-spectral imaging capabilities to facilitate the determination of treatment sub-areas with different skin conditions by the analysis module 102. Multi-spectral imaging relies on the differential reflectance of light from the skin to visualize and quantify, for example, blood flow and inflammation. Hemoglobin, the protein in red blood cells, plays an important role in this method. Hemoglobin absorbs light in specific parts of the spectrum, particularly in the visible red (600-700 nm) and green (500-600 nm) ranges. This absorption reduces the amount of light that is reflected back from areas with a high concentration of blood, such as inflamed or red areas of the skin. Multi-spectral imaging detects these changes in light reflectance, allowing it to visualize and quantify the areas of increased blood flow and vascular changes associated with skin inflammation.

When equipped with fluorescent and/or multi-spectral imaging capabilities, the image acquisition module 101 can directly output the area of interest that highlights a skin condition. This facilitates the analysis module 102 in determining treatment sub-areas associated with different skin conditions. As described elsewhere herein, image processing algorithms may be used to generate segmentation masks that enclose regions corresponding to specific skin conditions. Bounding boxes—such as axis-aligned or rotated minimum-area bounding boxes, as previously described—may then be generated based on these masks.

Consequently, the treatment sub-areas for different skin conditions can be readily determined by the analysis module 102 using the resulting masks and/or bounding boxes. Several image processing algorithms may be employed for this purpose. For example, thresholding converts grayscale images into binary images by applying a threshold value and is commonly used for background subtraction and basic object segmentation. Edge detection methods (e.g., Canny, Sobel) detect image boundaries by identifying regions with rapid intensity changes and are often used in object detection, feature extraction, and segmentation. Contour detection identifies the boundaries of objects in binary images and is applicable in shape analysis and segmentation tasks. Connected component labeling assigns unique labels to connected regions in binary images and is widely used in object detection and segmentation workflows. Watershed algorithms, which treat an image as a topographic surface, are useful for separating overlapping objects and performing complex region-based segmentation. K-means clustering, which may also be classified as an unsupervised machine learning method, partitions an image into k clusters based on color or intensity and is commonly used in color quantization and segmentation. GrabCut is an interactive foreground extraction algorithm used to separate objects from the background. Mean Shift Segmentation, another method that may be considered unsupervised learning, shifts each data point toward the mean of its neighborhood and is frequently used for segmentation and object tracking. Graph Cut Segmentation formulates the segmentation task as a graph-based optimization problem to identify the minimal cut and is widely used in high-quality image segmentation applications.

In some embodiments, the treatment module 103 may use light-based energy modalities—such as intense pulsed light (IPL), laser, or low-intensity light—for facial treatments. In such treatments, the treatment device 100 may determine treatment sub-areas based on the presence of different skin conditions. For example, sub-area 1 may exhibit skin inflammation, sub-area 2 may be characterized by pigmentation, and sub-area 3 may contain acne lesions. When the analysis module 102 determines that the current treatment sub-area is sub-area 1 (associated with skin inflammation), the analysis module 102 may command the treatment module 103 to emit red light in the range of 620-740 nm, as red light is known to help reduce inflammation and promote tissue healing. When the analysis module 102 determines that the current treatment sub-area is sub-area 2 (associated with pigmentation), the analysis module 102 may command the treatment module 103 to emit green light in the range of 495-570 nm. Green light has been shown to target melanocytes—the cells responsible for pigment production—thereby helping to break down existing melanin clusters and inhibit the formation of new ones. When the analysis module 102 identifies the current treatment sub-area as sub-area 3 (associated with acne), the analysis module 102 may command the treatment module 103 to emit blue light, typically centered around 465 nm. Blue light is known to induce an oxidative reaction in the skin, which kills acne-causing bacteria (e.g., Propionibacterium acnes) and reduces associated inflammation.

In some embodiments, treatment customization may include determining multiple sets of treatment sub-areas where each sub-area corresponds to a different anatomical classification criterion (e.g., skin thickness, depth of bone relative to skin surface, depth of muscle or nerve fiber relative to skin surface, orientation of muscle or nerve fiber, skin condition). To provide an example, assume treatment sub-areas are to be determined based on skin thickness and skin condition. In this example, the analysis module 102 may first divide the total facial treatment area into sub-areas based on skin thickness. Sub-areas with a thicker dermis may require higher RF output power, while sub-areas with a thinner dermis may require lower RF output power. These sub-areas may be delineated using key points, and the treatment module 103 may apply RF energy accordingly. In addition to the skin thickness-based classification, the analysis module 102 may determine treatment sub-areas associated with skin conditions—such as pigmentation, inflammation, or acne. Once such skin condition sub-areas are identified, the analysis module 102 may simultaneously command the treatment module 103 to deliver light-based energy appropriate for the sub-area. For example, red light (620-740 nm) may be simultaneously emitted in sub-areas with inflammation, green light (495-570 nm) in sub-areas with pigmentation, and blue light (˜465 nm) in sub-areas with acne. In these embodiments, the skin thickness-based sub-areas provide the foundational framework for customizing RF energy delivery, while the superimposed skin condition-based sub-areas dynamically trigger the addition of light-based modalities. The RF and light-based energies may be emitted concurrently through a co-located treatment head or integrated applicator, allowing simultaneous energy delivery. The analysis module 102 may coordinate both energy modalities in real time, adjusting parameters such as RF power level and light wavelength based on the overlapping sub-areas.

To provide a specific example, the total facial treatment area may be divided into sub-areas based on skin thickness and overlaid with sub-areas defined by localized skin conditions such as pigmented disorders. Referring to Error! Reference source not found., a thick area (treatment sub-area 107) is delineated by key points 12, 13, 14, and 15, representing a region of the face with relatively greater dermal thickness. Within this thick area lies a pigmented area (treatment sub-area 120) delineated by key points 70, 71, 72, and 73. The analysis module 102 may analyze incoming images from the image acquisition module 101 and determine the current treatment location based on the spatial relationship between the tissue-contacting part of the treatment device 100 and these defined sub-areas. When the analysis module 102 determines that the tissue-contacting part of the treatment device 100 falls within the pigmented area (e.g., within the boundary defined by key points 70, 71, 72, and 73), the analysis module 102 may command the treatment module 103 to simultaneously emit both green light—within the range of 495-570 nm—and RF energy. Green light is selected for its known efficacy in targeting melanin-producing melanocytes, thereby assisting in the treatment of pigmentation disorders. RF energy is concurrently delivered, customized for thick skin, to promote dermal remodeling and collagen stimulation. If, however, the analysis module 102 determines that the tissue-contacting part of the treatment device 100 lies within the thick area but outside the pigmented area, the analysis module 102 may command the treatment module 103 to emit only the RF energy appropriate for the skin thickness classification, without activation of the light-based modality.

One or more of the embodiments that follow involve the use of treatment indicators to customize treatment. The treatment indicators may include a dynamic treatment area indicator, an indicator based on a relative speed of the tissue-contacting part of the treatment device 100, an indicator relating to real-time skin reactions to treatment, a treatment contact quality indicator, and a temperature indicator.

In some embodiments, the treatment device 100 may include a treatment module 103 whose tissue-contacting part is required to remain in constant motion to cover a treatment area (or treatment sub-area) that is larger than the area of the tissue-contacting part itself. For example, in embodiments where the treatment module 103 utilizes RF as the energy modality for facial treatment, the tissue-contacting part is often required to continuously slide over the skin to distribute energy across the treatment area. In order to deliver a sufficient amount of RF energy to heat the tissue to a therapeutic temperature and maintain that temperature for a prescribed treatment time, the treatment area (or treatment sub-area) needs be optimized. If the treatment area (or sub-area) being treated is too large, the RF energy may become too dispersed, preventing the tissue from reaching the necessary therapeutic temperature, thereby compromising effectiveness. Conversely, if the treatment area (or sub-area) is too small, the RF energy may become overly concentrated, potentially elevating the tissue temperature beyond safe limits and introducing safety concerns.

In current state-of-the-art techniques, the determination of the appropriate treatment area relies on the operator's judgment and experience. However, this approach is inherently subjective and often inaccurate. To address this drawback and provide early feedback to operator regarding whether the treatment area is too large or too small, some embodiments described herein introduce a concept referred to as the dynamic treatment area, denoted as D(t). This may be defined as:

$$D ( t ) = \sum {S}_{i} ( t ) , and \\ {S}_{i} ( t ) = \{ \begin{matrix}0 , & if {S}_{i} is not treated during ( t - T , t ) \\ {A}_{i} , & if {S}_{i} is treated at least once during ( t - T , t )\end{matrix}$$

In this formulation, T represents the time interval over which the dynamic treatment area is defined, t denotes the current time, Sᵢ is a surface triangle on a 3D surface mesh, and Aᵢ is the area of that triangle.

In some embodiments, assume the treatment is an RF facial treatment. The intended treatment area is 40 cm², with a total treatment duration of 3 minutes. The area of the tissue-contacting part of the treatment device 100 (e.g., the electrode-covered region) is 4 cm², and the operator moves the treatment device 100 at a speed of approximately 3 cm/s. The time window T may range from 0.1 seconds to 3 minutes, preferably between 1 second and 1 minute, and most preferably between 3 and 10 seconds. If T is set too close to the total treatment time (e.g., 3 minutes), the dynamic treatment area can only be evaluated toward the end of the session, thus failing to provide early feedback. Conversely, if T is set too short (e.g., 0.1 seconds), the dynamic treatment area may not capture a meaningful pattern in the user's treatment behavior. When T is selected between 3 and 10 seconds, the dynamic treatment area provides a balanced and timely representation of the user's motion, allowing the system to offer early feedback if the area is determined to be too small or too large.

Continuing with this embodiment, and referring now to Error! Reference source not found.A, at t=0 seconds, the tissue contacting part of the device, which has a circular shape, is placed on the skin at position 121. At t=1.5, the tissue-contacting part is moved along the direction indicated by the arrow in Error! Reference source not found.B. Since T is 3 seconds, the dynamic treatment area at t=1.5 seconds includes all areas covered between t=0 seconds and t=1.5 seconds, as indicated by the shaded region. At t=3 seconds, the tissue-contacting part is moved along the direction indicated by the arrow in Error! Reference source not found.C, Since T is 3 seconds, the dynamic treatment area at t=3 seconds includes all areas covered between t=0 seconds and t=3 seconds, as indicated by the shaded region. At t=4.5 seconds, the tissue-contacting part is moved along the direction indicated by the arrow in Error! Reference source not found.D, Since T is 3 seconds, the dynamic treatment area at t=4.5 seconds includes all areas covered between t=1.5 seconds and t=4.5 seconds, as indicated by the shaded region. This pattern continues in Error! Reference source not found.E and Error! Reference source not found.F for t=6 and t=7.5 seconds, respectively, evaluating coverage over the most recent 3-second window.

A lower and upper threshold for the dynamic treatment area may be defined to establish a normal range. If the calculated dynamic treatment area D(t) falls outside this normal range, the analysis module 102 may generate warnings to alert the operator to take corrective action. These warnings may be communicated via the UI module 104 and may include, but are not limited to, voice prompts, textual messages, visual overlays, or other alert methods commonly understood by one of ordinary skill in the art. Additionally, the analysis module 102 may send commands to the treatment module 103 to interrupt the treatment. Such interruption may include turning off the device, pausing energy delivery, or reducing treatment intensity.

Continuing the above example, a lower threshold value of 20 cm² and an upper threshold value of 50 cm² may be selected for an intended treatment area of 40 cm². When the analysis module 102 determines that the dynamic treatment area D(t) falls below 20 cm², the analysis module 102 may alert the operator to expand the treatment coverage. When D(t) exceeds 50 cm², the system may warn the operator to reduce the area being treated. Additionally, the analysis module 102 may command the UI module 104 to display the current dynamic treatment area alongside the intended treatment area, enabling the operator to make informed and timely adjustments.

In some embodiments, the analysis module 102 may also dynamically adjust the RF output power to maintain a consistent power density across the treatment area. For example, if the configured RF power is 5 W over a current treatment area of 40 cm², the corresponding power density is 0.125 W/cm². This value may be used as a target power density during treatment. When the analysis module 102 determines that the dynamic treatment area is 35 cm², the analysis module 102 may command the treatment module 103 to decrease the RF power to 4.375 W, thereby maintaining the same power density of 0.125 W/cm². This real-time adjustment ensures that energy delivery remains within safe and effective limits, even as the operator deviates from the ideal treatment area.

To reconstruct the 3D surface mesh of the face and compute the surface triangles Sᵢ, the image acquisition module 101 may be equipped with hardware suitable for stereo vision, structured light, or photogrammetry, as previously disclosed in the present invention. The image acquisition module 101 may capture images for processing by the analysis module 102, which may apply stereo vision algorithms, structured light algorithms, or photogrammetry algorithms to perform 3D reconstruction of the facial surface.

In some embodiments, the energy-based aesthetic treatment device 100 may include a treatment module 103 whose tissue-contacting part is required to remain in constant motion to cover a treatment area (or treatment sub-area) larger than the area of the tissue-contacting part itself. For example, when the treatment module 103 uses RF for facial treatment, the tissue-contacting part shall slide continuously over the skin to distribute energy effectively. To ensure both safety and efficacy, the relative speed of the tissue-contacting part with respect to the skin must be optimized. If the tissue-contacting part is moved too quickly, excessive friction may be generated at the interface between the device and the skin, potentially causing epidermal damage. For example, rapid movement may increase the frictional force beyond a safe threshold, leading to irritation, burns, or micro abrasions. Additionally, high movement speeds may compromise the contact quality between the tissue-contacting surface and the skin. This is because at higher speeds, the operator may find it difficult to apply and maintain a consistent and appropriate amount of downward force, thereby resulting in intermittent or partial contact. Such degraded contact may reduce energy coupling efficiency and lead to uneven or ineffective treatment. Conversely, moving the tissue-contacting part too slowly may result in localized energy accumulation, which increases the risk of excessive tissue heating. This localized thermal build-up may exceed the intended therapeutic temperature and cause discomfort, burns, or thermal injury. A particularly hazardous scenario arises when the operator unintentionally leaves the tissue-contacting part stationary on the skin—often due to distraction or inattention, especially in at-home use settings. This complete lack of relative motion is typically contraindicated and can be dangerous, as the prolonged exposure of a single skin region to concentrated energy may result in serious adverse effects.

The current state-of-the-art treatment technique typically relies on the user or the operator to determine the appropriate movement speed of the tissue-contacting part of the device, often guided by their personal experience. However, this approach is inherently subjective and frequently inaccurate, leading to inconsistent treatment outcomes and potential safety concerns. To address this limitation, some embodiments described herein involve a method to calculate the relative speed of the tissue-contacting part of the device 101 with respect to the tissue being treated (e.g., the skin), based on vectorial velocity calculations. Specifically, the absolute velocity (with respect to the Earth) of both the tissue-contacting part of the device and the skin is first calculated in vector form. It is noted that both the tissue-contacting part and the skin may exhibit not only translational but also rotational motion. As a result, different points on either surface may have different instantaneous velocities. To manage this complexity and enable practical computation, the velocity of each surface may be approximated by averaging the velocities of several representative key points.

In some embodiments, if the tissue-contacting part includes a substantially square surface, its velocity v_{d} may be approximated by averaging the velocities of the four corner key points, such that:

$${v}_{d} = \frac{1}{4} ( {v}_{1} + {v}_{2} + {v}_{3} + {v}_{4} )$$

Likewise, the velocity of the skin vs may be calculated by averaging the velocities of representative key points associated with the current treatment area. For example, if the current treatment area is characterized by a triangular shape, the velocity may be approximated by averaging the velocities of the three corner key points, such that:

$${v}_{s} = \frac{1}{3} ( {v}_{1} + {v}_{2} + {v}_{3} )$$

In the context of the present invention, the term velocity refers to a three-dimensional vector quantity possessing both magnitude and direction, while the term speed refers to the scalar magnitude of the velocity. In some embodiments—particularly those where both the skin and the tissue-contacting part exhibit negligible rotational motion—this vectorial analysis may be simplified to scalar speed calculations without substantial loss of accuracy.

Without wishing to be bound by theory, other methods of approximating the velocity and/or speed of the tissue-contacting part and the skin may be employed, as would be understood by one of ordinary skill in the art. These alternative methods may include simplified vector projections, rigid-body approximations, center-of-mass tracking, or ML based estimations of motion vectors, all without departing from the spirit or scope of the principles of the present disclosure.

To calculate the velocity of the key points associated with the tissue-contacting part of the treatment device 100 and/or the skin, the three-dimensional coordinates—specifically, the x, y, and z positions of each key point with respect to a fixed Earth-centered reference frame—may be tracked over time. If the image acquisition module 101 captures images at a frame rate of N frames per second (fps), then the time interval Δt between consecutive frames may be defined as 1/N seconds. The velocity v of a representative key point may be calculated using its positional change over time as follows:

$$v = \frac{L 2 ( x 2 , y 2 , z 2 ) - L 1 ( x 1 , y 1 , z 1 )}{Δ t}$$

where L1 (x1,y1,z1) and L2 (x2,y2,z2) represent the 3D coordinates of the key point at the beginning and end of the interval Δt, respectively.

For this method to be effective, key points must be accurately identified and tracked across consecutive image frames. Several techniques may be employed to achieve this: 1) ML based algorithms may be utilized to detect key points on each image frame, as previously disclosed in the present invention. These algorithms may include neural network architectures trained to identify facial landmarks, anatomical features, or device boundaries; 2) Optical flow algorithms may also be employed to track key points. In this method, a key point is first identified in an initial frame, and its movement across subsequent frames is estimated based on local intensity gradients and motion coherence assumptions. This allows for efficient frame-to-frame tracking of motion.

Once the key points have been identified and tracked across frames, their 3D coordinates may be determined by projecting them onto a reconstructed 3D facial surface model, as previously disclosed in this invention. This projection enables spatial mapping of each key point within a consistent 3D reference geometry.

The relative velocity vᵣ of the tissue-contacting part with respect to the skin may then be computed as the vector difference between the device velocity v_{d} and the skin velocity vₛ:

$${v}_{r} = {v}_{d} - {v}_{s}$$

Subsequently, the relative speed Vᵣ—a scalar quantity representing the magnitude of the relative velocity vector—may be calculated as:

$${V}_{r} = \vert {v}_{r} \vert$$

This relative speed Vᵣ provides a direct and quantifiable metric for assessing the motion of the tissue-contacting part of the device with respect to the treatment area on the skin, and may be continuously monitored to determine whether the movement falls within a predefined safe and effective range.

In an alternative embodiment, the treatment device 100 (e.g., using analysis module 102) may bypass separate calculations of absolute velocity for the tissue-contacting part and the skin by directly computing the relative velocity based on averaged key point positions. At each frame, the analysis module 102 may first compute the average position of the key points on the tissue-contacting part of the device and separately compute the average position of the key points on the skin within the treatment area. The relative position vector may then be obtained by subtracting the average position of the skin key points from the average position of the tissue-contacting part key points. By performing this operation across two consecutive frames, the relative displacement may be calculated, and the relative velocity can be obtained by dividing this displacement by the time interval Δt between the two frames. The relative speed, which is the magnitude of the relative velocity vector, may then be readily determined. This method simplifies the computational process by treating each surface as a rigid body and relying on their average motion, which is particularly effective in embodiments where rotational effects are minimal or negligible. Without wishing to be bound by theory, other algorithms may also be employed to estimate the position of the skin or the tissue-contacting part. These may include, but are not limited to, centroid tracking, model fitting techniques, or machine learning-based pose estimation algorithms, without departing from the spirit or scope of the present invention.

A lower and upper threshold of the relative speed of the tissue-contacting part of the treatment device 100 with respect to the skin may be defined to establish a normal range. Once the analysis module 102 determines that the relative speed falls outside of this range, it may generate warnings to prompt the operator to take appropriate corrective actions. These warnings may be delivered via the UI module 104 and may include, but are not limited to, voice prompts, on-screen text messages, visual cues, or other warning modalities commonly understood by one of ordinary skill in the art. In certain embodiments, the analysis module 102 may also send commands to the treatment module 103 to interrupt the treatment. Such interruption may include turning off the device, pausing energy delivery, or reducing the treatment intensity.

As one exemplary embodiment for RF-based facial treatment, a lower threshold of 1 cm/s and an upper threshold of 5 cm/s may be selected to define the normal range for relative speed Vᵣ. When the analysis module 102 determines that Vᵣ<1 cm/s, it may alert the operator to move the tissue-contacting part faster. Conversely, when Vᵣ>5 cm/s, the system may issue a warning to reduce the movement speed. Additionally, the analysis module 102 may command the UI module 104 to display the real-time value of the relative speed Vᵣ along with its normal range, thereby enabling the operator to make timely and informed adjustments during the treatment.

In some embodiments, a moving average algorithm may be applied to the relative speed Vᵣ before evaluating whether it falls within the normal range. This temporal averaging may help smooth out transient spikes or dips in motion and reduce unnecessary or momentary warning signals, particularly those resulting from short-term fluctuations that are not clinically significant. For RF facial treatments, the moving average may be calculated over a time window ranging from 0.1 seconds to 1 minute, more preferably between 1 second and 10 seconds. For example, with a 3-second moving average window and a system update frequency of 10 Hz, the moving average array would consist of 30 successive Vᵣ values, continuously refreshed to reflect the most recent treatment behavior.

In some embodiments, real-time skin reactions (or more broadly, tissue reactions) may develop during the delivery of energy in an energy-based aesthetic treatment. These reactions may include, but are not limited to, skin redness (erythema) and swelling (edema). For the treatment to remain safe, comfortable, and effective, it is critical to ensure that such tissue responses remain within normal range throughout the course of energy delivery. For example, individuals with sensitive skin may have a lower tolerance for RF energy compared to those with normal skin. In such cases, excessive redness may develop rapidly during treatment. If this reaction goes undetected and the treatment continues unabated, the risk of post-treatment adverse effects—such as prolonged erythema, irritation, or even burns—may increase significantly. Similarly, if the treatment results in noticeable swelling and this condition is not identified and addressed in real-time, continued energy delivery may further exacerbate tissue trauma, again elevating the likelihood of adverse post-treatment outcomes.

In the present invention, a tissue reaction within normal range refers to a level of physiological response that is anticipated and considered safe for a specific type of energy-based aesthetic treatment. In some embodiments, the extent of skin redness (erythema) may serve as an indicator to determine whether the tissue reaction remains within normal range during the course of treatment. In other embodiments, the extent of skin swelling (edema) may be used as the primary indicator for assessing whether the tissue is reacting within normal range. In the context of energy-based aesthetic treatments, excessive skin redness and/or swelling is often indicative of overtreatment. When these reactions exceed the expected physiological threshold, the probability of adverse events—including post-treatment discomfort, inflammation, or tissue damage—increases significantly. Accordingly, real-time monitoring of such reactions is essential to ensure both safety and treatment efficacy.

The current approach relies heavily on the trained operator to visually assess immediate skin reactions during the delivery of treatment. However, such assessments are often highly subjective and can vary significantly between individuals. As a result, the accuracy and consistency of detecting abnormal tissue responses may be compromised, potentially leading to delayed intervention and an increased risk of adverse treatment outcomes.

To assess skin redness in real time, the image acquisition module 101 may include visible light digital camera(s), such as digital CCD or CMOS cameras, which may be positioned to capture both the tissue-contacting part of the treatment device 100 and the treatment area (or treatment sub-area, if used in conjunction with previously disclosed embodiments). In some embodiments, the image acquisition module 101 may further include multi-spectral imaging capabilities to enhance the detection and quantification of skin redness. Multi-spectral imaging operates based on the differential reflectance of light from the skin and is particularly effective in visualizing and quantifying the distribution of chromophores such as hemoglobin, a protein found in red blood cells. Hemoglobin exhibits characteristic absorption in specific spectral bands—most notably in the visible red (600-700 nm) and green (500-600 nm) wavelength ranges. In areas with elevated blood concentration, such as regions of inflammation or erythema, this absorption reduces the amount of light reflected back to the imaging sensor. By capturing and analyzing these reflectance changes across multiple spectral bands, multi-spectral imaging enables the analysis module 102 to visualize and quantify skin redness, thereby facilitating real-time assessment of tissue reaction during treatment.

The captured images are transmitted to the analysis module 102 for further analysis. According to various embodiment, a skin redness score R may be computed as a weighted sum of the redness area and the redness intensity:

$$R = a A + bI$$

where A represents the area within which skin redness is detected, I denotes the redness intensity, and a and b are the respective weighting factors for A and I.

To determine whether the treatment should be halted or terminated in order to minimize the risk of adverse effects, the analysis module 102 may evaluate the redness score R against one or more threshold values. Two methods may be employed for threshold determination: (1) an absolute increase in the redness score, denoted as Rₐ, and (2) a percentage increase, denoted as Rₚ. The absolute increase Rₐ may be calculated as:

$${R}_{a} = {R}_{r} - {R}_{b}$$

The percentage increase Rₚ may be calculated as:

$${R}_{p} = \frac{{R}_{r}}{{R}_{b}} \times 100 \%$$

where R_{b} is the baseline redness score computed prior to the commencement of treatment, and Rᵣ is the real-time or periodically computed redness score during treatment (e.g., computed at a rate >10 frames per second or at fixed time intervals such as every 1 second or 30 seconds). The analysis module 102 may determine that treatment should be interrupted or discontinued once either Rₐ is greater than or equal to an absolute threshold Rₜₐ, or Rₚ is greater than or equal to a percentage threshold Rₜₚ.

To compute the redness score R from each image, the corresponding redness area A and redness intensity I shall first be calculated. One approach to extract these values may involve the use of ML based algorithms. Specifically, the training dataset may include pixel-wise labeled redness regions as ground truth. The ML based algorithms may then be trained to perform pixel-wise segmentation of the redness areas, from which the area A can be computed based on the total number of segmented pixels. Additionally, the training dataset may include intensity labels, such as redness severity levels on a predefined scale (e.g., 0 to 10, where 0 represents no detectable redness and 10 represents the most severe redness). The ML based algorithm may be trained to perform classification to assign an appropriate redness intensity score/to each image. In some embodiments, the ML based algorithms may simultaneously output both segmentation masks for area calculation and intensity scores, enabling an efficient and unified pipeline for real-time redness evaluation.

Another method to calculate redness area A and redness intensity I may involve image processing algorithms. According to some embodiments of the present invention, the image acquisition module 101 may captures RGB images of the skin. Several image processing algorithms may help in enhancing, segmenting, and analyzing the incoming RGB images to provide a clear visualization of skin redness, for example, thresholding can be applied to the intensity values of the incoming images to isolate regions where hemoglobin absorption is significant. By setting a threshold value, pixels with intensity values above or below this threshold can be identified as areas of redness. This binary segmentation helps in distinguishing red regions from the rest of the skin, providing a clear map of skin redness distribution. Hue, Saturation, and Value (HSV) Color Space is another image processing algorithm used for detecting redness. Skin redness can be effectively highlighted by converting the image from the RGB color space to the HSV color space. In this space, the hue component represents the color information, which can be specifically tuned to identify red hues associated with increased blood flow. By focusing on the hue values corresponding to redness and combining them with saturation and value thresholds, areas of skin redness can be accurately extracted and visualized. Watershed Algorithm is yet another image processing algorithm for segmenting red regions in an image, particularly useful when dealing with overlapping or touching objects. When applied to the incoming images of skin, the watershed algorithm can help in delineating the boundaries of red areas by treating the intensity gradient of the image as a topographic surface. The algorithm floods this surface from the markers placed in the regions of interest, such as the areas identified by thresholding or HSV analysis, and segments the image into distinct regions. This method is particularly useful for accurately defining the edges of inflamed or red areas on the skin. Canny Edge Detection is yet another image processing algorithm that could be used to detect skin redness. The Canny edge algorithm can be applied to identify the boundaries of red regions by detecting sharp changes in intensity. This technique involves multiple steps, including gradient calculation, non-maximum suppression, and edge tracking by hysteresis, which together help in highlighting the contours of red areas. By combining edge detection with other segmentation methods, a more precise localization of skin redness can be achieved. Without wishing to be bound by theory, other image processing algorithms may be employed as well, as commonly understood by one of ordinary skill in the art to which the invention pertains.

In some embodiments, the calculation of the redness area A and redness intensity I based solely on two-dimensional (2D) images may introduce inaccuracies due to perspective distortion, non-uniform lighting, or variations in camera angle and distance. To improve the precision of these calculations, the present invention may utilize the 3D reconstructed face model, onto which the detected redness regions from the 2D images are mapped. As previously disclosed, the image acquisition module 101 may include hardware suitable for 3D reconstruction, such as stereo vision, structured light, or photogrammetry, and the analysis module 102 may implement algorithms to project the segmented 2D redness regions onto the corresponding regions of the 3D surface mesh. Once mapped, the redness area A may be computed based on the actual surface geometry, and the redness intensity I may be evaluated with improved spatial and photometric accuracy. This 3D-based approach allows for more consistent and anatomically accurate assessment of skin reaction during treatment, particularly in curved or occluded facial regions where 2D projections alone may be unreliable.

If the analysis module 102 determines that either Rₐ>Rₜₐ or Rₚ>Rₜₚ is satisfied, it may generate warnings to the operator, prompting them to take appropriate corrective actions. Such warnings may be communicated through the UI module 104 and may include, but are not limited to, voice prompts, text messages, visual indicators, or other common alert mechanisms readily understood by one of ordinary skill in the art. In response to the detected condition, the analysis module 102 may also transmit commands to the treatment module 103 to interrupt the treatment. This interruption may involve turning off the device, pausing energy delivery, or reducing the treatment intensity to mitigate further adverse tissue reactions. Conversely, if the analysis module 102 determines that the tissue reaction remains within the normal range, it may command the treatment module 103 to continue the treatment.

To assess skin swelling in real time, the image acquisition module 101 may be equipped with hardware configurations suitable for stereo vision, structured light, or photogrammetry, as previously disclosed in the present invention. The image acquisition module 101 may acquire images of the treatment area and transmit them to the analysis module 102 for 3D reconstruction of the region of interest. As described in earlier sections, the analysis module 102 may implement stereo vision algorithms, structured light algorithms, or photogrammetry algorithms to construct an accurate 3D surface model of the skin. Alternatively, the 3D reconstruction may be performed using ML based algorithms that estimate depth information from a single RGB image, as also previously disclosed. These approaches enable the system to capture topological changes in the skin surface, such as swelling or protrusions, in a time-resolved and spatially accurate manner.

According to various embodiments of present invention, a skin swelling score S may be computed to quantify the extent of swelling during treatment. The skin swelling score S may be defined as a weighted sum of the swelling area and swelling volume:

S=aA+bV

where A represents the area within which swelling is detected, V denotes the swelling volume, and a and b are weighting factors for area and volume, respectively. This formulation allows the system to account for both the spatial extent and the severity of the swelling when evaluating tissue reaction.

A threshold swelling score Sₜ may be stored in the analysis module 102 to establish a limit above which treatment should be halted or terminated to minimize the risk of adverse effects. The swelling score Sₜ may be computed in real time (e.g., at frame rates greater than 10 fps) or at specific time intervals (e.g., every 1 second or every 30 seconds). The analysis module 102 may determine that the treatment should be interrupted or stopped when the condition S>Sₜ is satisfied. This enables timely intervention and enhances treatment safety by preventing continued energy delivery to areas exhibiting abnormal tissue response.

To determine the skin swelling score S associated with each image, the corresponding swelling area A and swelling volume V shall be calculated. The swelling volume V may be determined by calculating the volumetric change between the 3D model of the treatment area reconstructed prior to the commencement of treatment and the 3D model reconstructed during the treatment. As one exemplary embodiment, if the volume of the treatment area before treatment is denoted as V_{b}, and the volume during treatment is denoted as Vᵣ, then the swelling volume V may be calculated as:

$$V = {V}_{r} - {V}_{b}$$

Several algorithms, as previously disclosed in detail in the present invention, may be employed by the analysis module 102 to compute the swelling volume V. These algorithms may include, but are not limited to, 3D mesh comparison techniques (e.g., iterative closest point alignment, volumetric differencing) and ML based algorithms trained to estimate localized volumetric changes. Once V is calculated, the swelling area A may be computed by the analysis module 102 by summing the surface area over regions where swelling is detected, based on geometric or visual deviation from the baseline 3D model.

If the analysis module 102 determines that the swelling score exceeds the threshold, e.g., S>Sₜ, it may generate warnings to the operator, prompting them to take appropriate corrective actions. These warnings may be delivered through the UI module 104 and may include, but are not limited to, voice prompts, on-screen messages, visual highlights, or other signaling mechanisms commonly understood by one of ordinary skill in the art. In response, the analysis module 102 may also issue commands to the treatment module 103 to interrupt the treatment, which may include turning off the device, pausing energy delivery, or reducing the treatment intensity to prevent further adverse tissue response. Conversely, if the analysis module 102 determines that the tissue reaction remains within the normal range, it may command the treatment module 103 to continue the treatment.

Without wishing to be bound by theory, another illustrative example involves ablative skin laser treatments, where the primary endpoint is typically characterized by the appearance of lesions exhibiting specific color and/or size. In such cases, close visual monitoring of these primary indicators—such as lesion shape, and hue—is important to ensure that the treatment remains within a safe and therapeutically effective boundary.

In some embodiments, the energy-based aesthetic treatment device 100 may include a treatment module 103 whose tissue-contacting part is required to be in full contact with the skin or sufficiently coupled to the skin to ensure effective and safe energy delivery. For example, in RF treatments, the electrodes (e.g., the tissue-contacting part) shall maintain full contact with the skin. In ultrasound treatments, the ultrasound transducer (e.g., the tissue-contacting part) shall be adequately coupled to the skin surface. Inadequate contact in either case can lead to undesirable effects—for instance, partial electrode contact may result in localized current concentration, potentially creating hot spots and thermal injury, while insufficient ultrasound coupling may lead to acoustic energy reflection, diminishing treatment efficacy and increasing the risk of tissue damage.

In current practice, it is the responsibility of the operator to ensure that proper contact is consistently maintained throughout the treatment. However, this requirement for constant vigilance can be burdensome, especially for laypersons using the device in at-home settings, where distractions are common and expertise is limited.

To address these limitations and provide real-time feedback on the contact quality of the tissue-contacting part, the image acquisition module 101 may be equipped with one or more visible light digital cameras, such as CCD or CMOS sensors. These cameras may be oriented to simultaneously capture both the tissue-contacting part of the treatment device 100 and the treatment area. The captured images may be transmitted to the analysis module 102 for further analysis. The analysis module 102 may analyze one or more image frames to determine whether treatment quality requirements—specifically, proper contact—are being met during the procedure.

According to some embodiments of the present invention, to evaluate the adequacy of contact between the tissue-contacting part of the treatment device 100 and the tissue being treated, a contact quality indicator Q may be defined as:

$$Q = \frac{{A}_{c}}{{A}_{t}} \times 100 \%$$

where A_{c} represents the area of the tissue-contacting part that either physically contacts or is sufficiently coupled to the tissue, and Aₜ represents the total area of the tissue-contacting part.

When the analysis module 102 determines that the contact quality indicator Q falls below a threshold value Qₜ, it may generate warnings to the operator, prompting them to take appropriate corrective actions. Such warnings may be communicated through the UI module 104, including, but not limited to, voice prompts, text alerts, visual guidance, or other warning signals commonly understood by one of ordinary skill in the art. The analysis module 102 may also send commands to the treatment module 103 to interrupt the treatment, such as powering off the device, pausing energy delivery, or reducing the treatment intensity.

In some embodiments, the analysis module 102 may also display the contact quality indicator Q in real time to provide the operator with continuous feedback regarding the adequacy of tissue contact. In these embodiments, the UI module 104 may be configured to visualize the spatial distribution of contact across the tissue-contacting part of the treatment device 100. For example, the UI module 104 may present a visual overlay or schematic representation of the tissue-contacting surface, highlighting regions with inadequate contact using a distinct color such as red, while adequately coupled regions may be shown in green. This real-time visual guidance enables the operator to promptly identify which portion(s) of the tissue-contacting part require repositioning or adjustment. In some embodiments, the contact quality indicator Q may also be displayed numerically as a percentage, continuously updated during the treatment process. By combining quantitative metrics with intuitive visual cues, the system enhances user understanding and facilitates timely corrective actions to ensure consistent treatment quality and safety.

In some embodiments, the contact quality indicator Q may also be used to dynamically adjust the treatment intensity or output power of the treatment module 103 to maintain a consistent power density relative to the surface area of the tissue-contacting part that is in contact or sufficiently coupled with the tissue. In these embodiments, the analysis module 102 may calculate the power density as the treatment output power divided by the effective contact area (e.g., A_{c}). To maintain a constant target power density, the analysis module 102 may proportionally scale the treatment intensity or output power based on the value of Q. For instance, when the contact quality indicator Q is determined to be 80%, the analysis module 102 may command the treatment module 103 to reduce the treatment intensity to 80% of its intended value. Likewise, when Q is determined to be 50%, the treatment intensity may be reduced to 50%. This proportional adjustment ensures that energy delivery per unit area remains consistent, thereby minimizing the risk of overexposure in localized regions and enhancing the safety and efficacy of the treatment.

The analysis module 102 may implement ML based algorithms trained to infer the contact quality directly from image data. In one example, the training dataset may consist of labeled images categorized as “full contact” or “sufficiently coupled,” and “partial contact” or “insufficiently coupled,” thereby treating the task as a binary classification problem. Alternatively, images may be labeled with graded levels of contact, such as “full contact,” “half contact,” or “quarter contact,” turning the task into a multiclass classification problem to achieve finer granularity in the assessment of contact quality.

The analysis module 102 may also employ ML based key point detection algorithms to locate anatomical landmarks or device contours. As previously disclosed in this invention, the spatial relationship between key points corresponding to the tissue-contacting part and those corresponding to the skin may be analyzed to infer the extent of contact.

Without wishing to be bound by theory, other ML based approaches, as commonly known to those skilled in the art, may also be employed. For instance, semantic segmentation algorithms may be used to independently segment the tissue-contacting part of the device and the tissue being treated. The spatial overlap or proximity between the segmented areas can then be analyzed by the analysis module 102 to determine contact quality.

In some embodiments, the analysis module 102 may additionally utilize sensor data to quantify the contact quality indicator Q. Suitable sensors may include, but are not limited to, force-sensitive resistors, capacitive touch sensors, piezoelectric sensors, optical sensors (e.g., for reflectance or beam interruption), and pressure sensors. These sensors are preferably mounted on or near the tissue-contacting surface of the device, either as a complement to or substitute for the image acquisition module 101, to support accurate determination of the contact quality.

In some embodiments, energy-based aesthetic treatments are designed to deliver therapeutic energy to the tissue in a manner that raises the tissue temperature to a target level and maintains that temperature for a predetermined duration to achieve the intended therapeutic effect. As previously disclosed in the present invention, several treatment indicators—either independently or in combination—may influence the tissue temperature during treatment. These include, but are not limited to, the dynamic treatment area, the relative speed of the tissue-contacting part with respect to the tissue being treated, and the contact quality between the tissue-contacting part and the tissue. Conventional solutions rely on the operator to subjectively monitor and maintain these treatment indicators within an optimal range. However, this approach imposes a significant burden on the operator and is particularly impractical in at-home settings where the user may lack professional training or attentiveness. In practice, one or more parameters may deviate from the acceptable range and remain unnoticed, potentially compromising both the safety and effectiveness of the treatment.

To address these limitations and provide real-time feedback regarding deviations in critical parameters, the image acquisition module 101 may be equipped with an infrared thermography camera (IR camera) in addition to the imaging components used to monitor dynamic treatment area, relative speed, and contact quality, as previously described. The IR camera enables the image acquisition module 101 to capture thermal images of the treatment area, which are subsequently analyzed by the analysis module 102 to determine the tissue temperature T, such as the skin surface temperature in the context of RF facial treatments.

Without wishing to be bound by theory, other temperature monitoring techniques may also be utilized in conjunction with, or as alternatives to, IR imaging. For example, contact-based temperature sensors, such as thermistors mounted on the surface of the tissue-contacting part, or non-contact infrared thermometers, may be employed. These sensors may be integrated into the image acquisition module 101 or deployed separately to enhance the robustness of temperature monitoring.

The analysis module 102 may compare the measured tissue temperature T against a reference temperature or acceptable temperature range. For instance, a skin temperature range of approximately 42° C. to 44° C. is commonly used for RF facial treatments. When the analysis module 102 determines that the tissue temperature T falls outside the target range, it may initiate diagnostic procedures to identify which treatment indicators are contributing to the deviation. Monitoring the tissue temperature continuously and invoking the check on other treatment indicators only when the temperature is abnormal can be advantageous, as the evaluation of those parameters—such as dynamic treatment area, relative speed, and contact quality—may introduce additional hardware and software overhead. This strategy reduces computational burden and system complexity while still enabling effective and timely feedback to maintain treatment safety and efficacy.

In some embodiments, the dynamic treatment area may influence the tissue temperature T. Upon determining that the dynamic treatment area is outside its acceptable bounds—as previously disclosed in the present invention—the analysis module 102 may generate alerts. For example, if the dynamic treatment area exceeds its upper threshold, the operator may be advised to reduce the area being treated. Conversely, if the dynamic treatment area is below the lower threshold, the operator may be prompted to expand the coverage. These prompts may be delivered via the UI module 104, and the analysis module 102 may also command the UI module 104 to display both the current dynamic treatment area and the intended treatment area to support real-time decision-making. In addition, analysis module 102 may dynamically adjust the RF output power to maintain a consistent power density based on the determination of the dynamic treatment area and the treatment area, as described elsewhere herein.

In some embodiments, the relative speed of the tissue-contacting part with respect to the tissue being treated may also influence the tissue temperature T. When the analysis module 102 determines that the relative speed is outside the predefined optimal range—as previously disclosed—it may issue warnings to the operator through the UI module 104. These warnings may include voice prompts, textual messages, visual guidance, or other user-friendly alert mechanisms. Furthermore, the analysis module 102 may command the treatment module 103 to interrupt the treatment. Such interruption may include turning off the device, pausing the treatment, or reducing the treatment intensity.

In some embodiments, the contact quality may influence tissue temperature T. When the analysis module 102 determines that the contact quality indicator Q is below a threshold Qₜ, as previously described, it may issue warnings to the operator through the UI module 104. These warnings may include voice prompts, textual messages, visual guidance, or other user-friendly alert mechanisms. Furthermore, the analysis module 102 may command the treatment module 103 to interrupt the treatment. Such interruption may include turning off the device, pausing the treatment, or reducing the treatment intensity.

According to other embodiments, the analysis module 102 may also display the contact quality indicator Q in real time to provide the operator with continuous feedback regarding the adequacy of tissue contact. In these embodiments, the UI module 104 may be configured to visualize the spatial distribution of contact across the tissue-contacting part of the treatment device 100. For instance, the UI module 104 may present a visual overlay or schematic representation of the tissue-contacting surface, highlighting regions with inadequate contact using a distinct color such as red, while adequately coupled regions may be shown in green. This real-time visual guidance enables the operator to promptly identify which portion(s) of the tissue-contacting part require repositioning or adjustment. In some embodiments, the contact quality indicator Q may also be displayed numerically as a percentage, continuously updated during the treatment process. By combining quantitative metrics with intuitive visual cues, the system enhances user understanding and facilitates timely corrective actions to ensure consistent treatment quality and safety.

In some embodiments, a method of customizing an energy-based aesthetic treatment is provided. The method may incorporate one or more aspects relating to treatment indicators discussed herein. The method may be implemented using treatment device 100. In some embodiments, the method may include obtaining image data identifying a treatment area and a tissue-contacting part of the treatment device. In some embodiments, the method may include calculating a dynamic treatment area representing a surface area of tissue that has been treated within a time window that is prior to a current time based on a location of the tissue-contacting part of the treatment device relative to the treatment area during the time window. In some embodiments, the method may further include determining whether the dynamic treatment area is larger than an upper bound threshold treatment area size or lower than a lower bound threshold treatment area size. In some embodiments, the method may further include performing one or more actions based on the dynamic treatment area being larger than the upper bound threshold treatment area size or lower than the lower bound threshold treatment area size. The one or more actions may include at least one of: adjusting at least one treatment setting of the one or more treatment settings, and generating and providing a warning or recommendation for display on a user interface accessible by an operator of the treatment device.

Additional embodiments follow which are described as part of the method discussed above. Notably, each of the embodiments that follow may be part of a separate method (e.g., a process excluding the calculation of the dynamic treatment area).

In some embodiments, the method may further include determining a relative speed of the tissue-contacting part with respect to the treatment area based on motion analysis of key points on the tissue-contacting part and the treatment area.

In some embodiments, the tissue-contacting part of the treatment device may be smaller than the treatment area and may deliver energy by traversing the treatment area while the energy-based aesthetic treatment is being performed. In these embodiments, the method may further include calculating, over a time window and while traversing the treatment area during the energy-based aesthetic treatment, a relative speed of the tissue-contacting part of the treatment device based on positions of one or more key points associated with the tissue-contacting part and one or more key points associated with a surface of the treatment area. The method may further include determining whether the relative speed is greater than an upper bound threshold speed or lower than a lower bound threshold speed. The method may further include performing one or more actions based on the relative speed being greater than the upper bound threshold or lower than the lower bound threshold speed. The one or more actions include at least one of: adjusting at least one treatment setting of the one or more treatment settings, and generating and providing a warning or recommendation for display on a user interface accessible by an operator of the treatment device.

In some embodiments, the method may further include calculating a moving average of the relative speed over the time window. In this embodiment, the determination as to whether the relative speed is greater than the upper speed threshold or less than the lower speed threshold may be based on the moving average.

In some embodiments, the method may further include determining a skin reaction to a treatment sub-area. In this embodiment, the method may further include determining a reaction intensity level of the skin reaction. In this embodiment, the method may further include determining a reaction score based on a weighted sum of a region of tissue corresponding to the skin reaction and the reaction intensity level. In this embodiment, the method may further include determining whether the reaction score is greater than or equal to a permissible tissue reaction threshold. In this embodiment, the method may further include performing one or more actions based on the redness score being greater than or equal to the permissible tissue reaction threshold. The one or more actions include at least one of: adjusting at least one treatment setting of the one or more treatment settings, and generating and providing a warning or recommendation for display on a user interface accessible by an operator of the treatment device. The reaction sub-area may refer to redness and/or swelling. The reaction score may refer to a redness score and/or a swelling score.

In some embodiments, the method may further include determining a contact quality indicator that represents a percentage of the tissue-contacting part of the treatment device that is in physical contact with a surface of the region of tissue included in the current treatment sub-area. In this embodiment, the method may further include determining whether the contact quality indicator is less than a contact quality threshold value. In this embodiment, the method may further include performing one or more actions based on the contact quality indicator being less than the contact quality threshold value. The one or more actions may include at least one of: adjusting at least one treatment setting of the one or more treatment settings, and generating and providing a warning or recommendation for display on a user interface accessible by an operator of the treatment device.

In some embodiments, adjusting at least one treatment setting includes adjusting a treatment intensity level or output power level such that the treatment intensity level or the output power level is proportionally reduced based on the percentage of the tissue-contacting part of the treatment device that is in physical contact with the surface of the region of tissue included in the current treatment sub-area.

In some embodiments, the method may further include measuring a tissue temperature at the current treatment area. In this embodiment, the method may further include determining that the tissue temperature exceeds a threshold temperature value. In this embodiment, the method may further include determining a treatment performance indicator contributing to the tissue temperature exceeding the threshold temperature value. The treatment performance indicator may be one of a dynamic treatment area, a relative speed of the tissue-contacting part of the treatment device, or a contact quality indicator. In this embodiment, the method may further include determining whether the treatment performance indicator satisfies a treatment performance threshold. In this embodiment, the method may further include performing one or more actions based on the treatment performance indicator satisfying the treatment performance threshold. The one or more actions may include at least one of: adjusting at least one treatment setting of the one or more treatment settings, and generating and providing a warning or recommendation for display on a user interface accessible by an operator of the treatment device

Some embodiments described below involve pre-treatment personalization of an energy-based aesthetic treatment. Prior to the commencement of an energy-based aesthetic treatment, personalized treatment planning is of critical importance. Unlike conventional approaches that apply a uniform treatment plan across all users, the present invention recognizes that each individual possesses unique anatomical, physiological, and pathological characteristics that may necessitate tailored treatment plans. For instance, variability in skin thickness, hydration level, pigmentation, and fat distribution can all influence treatment outcomes and safety. In some cases, pre-existing skin conditions may present contraindications to specific energy modalities. For example, in RF facial treatments, it is generally contraindicated to treat sunken regions of the face where subcutaneous fat has been lost due to aging, medication, or prior interventions. Delivering RF energy in such areas may result in additional fat degradation, potentially worsening the sunken appearance and resulting in aesthetically undesirable or clinically adverse outcomes. Similarly, the presence of inflamed or irritated skin may exacerbate inflammatory responses or cause significant discomfort if treated. Therefore, precise identification and appropriate exclusion of certain anatomical regions from energy delivery is essential to ensuring the safety and efficacy of treatment.

In some embodiments, the image acquisition module 101 may be equipped with hardware configurations suitable for stereo vision, structured light, or photogrammetry, as previously disclosed in the present invention. These configurations enable the image acquisition module 101 to capture image data necessary for the analysis module 102 to perform 3D facial reconstruction. Depending on the embodiment, the analysis module 102 may implement stereo vision algorithms, structured light processing algorithms, or photogrammetry algorithms to reconstruct a 3D model of the user's face. Alternatively, ML based algorithms capable of estimating depth from single RGB images may also be employed, as previously disclosed.

Once a 3D model of the user's face has been reconstructed, the analysis module 102 may analyze this model to identify regions exhibiting abnormal depressions or contours that deviate from normative facial geometry. In some embodiments, a reference 3D facial model may be stored in memory or accessed from a remote database. This reference model may be based on statistical averages of age-matched, gender-matched, BMI-matched individuals without significant facial volume loss. The analysis module 102 may then perform a geometric comparison between the reconstructed user model and the reference model. Algorithms such as iterative closest point (ICP) or mesh registration techniques may be used to align the two models. After alignment, the severity of the depression may be estimated.

As an exemplary embodiment, a depression score D may be computed as a weighted sum of the depression area A and depression volume V:

$$D = a A + b V$$

where A represents the surface area within which depression is detected, V represents the volume loss in that region, and a, b are weighting coefficients selected based on clinical relevance or empirical tuning. A threshold depression score Dₜ may be defined, above which the region is flagged as a sunken area. For instance, if D>Dₜ, the analysis module 102 may designate the region as unsuitable for energy delivery. The depression volume V may be calculated based on volume loss between the user's 3D facial model and the reference model. As an exemplary embodiment:

$$V = {V}_{b} - {V}_{r}$$

In the equation above, V_{b} denotes the reference model's volume over a region of interest, and Vᵣ denotes the user's volume over the same region. This calculation ensures that volume loss (e.g., when V_{b}>Vᵣ) yields a positive V. Several algorithms, as previously disclosed in the present invention, may be employed by analysis module 102 to calculate V, including but not limited to: 3D mesh comparison algorithms and ML-based algorithms. Once V is calculated, A can be calculated by the analysis module 102 by summing up the surface area of where depression is detected.

If the analysis module 102 determines D>Dₜ is satisfied, the analysis module 102 may generate a personalized treatment plan that omits these areas from energy delivery. In accordance with various embodiments, the UI module 104 may communicate this plan to the operator. The UI module 104 may visually highlight the sunken regions on the user's 3D facial map, overlaid with boundaries that indicate exclusion zones. In one embodiment, the UI module 104 may display these regions in a distinct color (e.g., red or orange) to indicate that they are not suitable for treatment. In another embodiment, the UI module 104 may provide visual, textual or voice feedback during the treatment, notifying the user that specific areas are excluded from the planned treatment due to risk of adverse outcomes.

In some embodiments, facial regions exhibiting mild volume loss—e.g., the depression score D does not exceed the threshold value of Dₜ for classification as a sunken area—may still be included in the personalized treatment plan with appropriate cautionary adjustments. For instance, in the context of RF facial treatments, certain regions of the face may exhibit moderate depressions that suggest partial fat loss but do not present a direct contraindication for treatment, then the condition may be labeled as a mild sunken area. In these embodiments, the analysis module 102 may allow treatment to proceed with caution and may generate specific guidance as part of the personalized treatment plan. Such guidance may include but is not limited to: (1) instructing the treatment module 103 to use a shallower electrode configuration, such that the RF energy is confined shallower in the dermis and does not penetrate deeply enough to affect the underlying fat layer; and (2) lowering the treatment intensity level (or RF output power) when the analysis module 102 detects that the user is currently treating the identified mild sunken area. These modifications allow treatment to continue while reducing the risk of exacerbating fat loss, thereby balancing safety with therapeutic efficacy in anatomically sensitive areas.

Personalized treatment planning, as disclosed in the present invention, may further include the inclusion or exclusion of risk areas from energy-based aesthetic treatments. Certain skin conditions may pose increased safety risks or diminish treatment effectiveness when exposed to energy modalities such as radiofrequency, ultrasound, laser, intense pulsed light (IPL), or low-level light therapy. Among these conditions, inflamed skin, infected lesions, and acne-prone regions are of particular concern. Inflamed skin may exhibit heightened sensitivity and vascular reactivity, making it more susceptible to excessive heating, pain, or post-treatment erythema. Infected skin or open lesions, including those associated with acne, may harbor pathogenic bacteria that could spread or worsen under thermal or mechanical stimulation. Moreover, acne nodules or pustules may rupture if subjected to inappropriate energy delivery, potentially causing scarring or secondary infection. Therefore, accurate identification and exclusion of such risk areas is important for maintaining safety and optimizing treatment outcomes. Other skin conditions that may also be assessed for their risk severity include, but are not limited to, eczema, psoriasis, rosacea, broken capillaries, and post-procedural wounds.

In accordance with various embodiments of the present invention, the image acquisition module 101 may be configured to facilitate the detection of skin conditions that constitute risk areas prior to treatment. Several imaging modalities may be employed either individually or in combination. In one embodiment, visible light imaging using a standard RGB digital camera may be used to capture high-resolution images of the treatment area. These images are transmitted to the analysis module 102, which analyzes color and texture patterns using image processing algorithms and/or ML based algorithms to detect signs of inflammation such as skin redness. In another embodiment, multi-spectral imaging may be employed. As previously disclosed in the present invention, hemoglobin exhibits distinct absorption properties in the visible spectrum, particularly within the green and red wavelengths. By analyzing differential light reflectance across multiple spectral bands, the image acquisition module 101 can highlight areas of increased blood flow associated with inflammation. These spectral images are then processed by the analysis module 102 to localize vascular-rich zones indicative of inflammation.

According to some embodiments, fluorescent imaging capabilities may also be utilized to detect infected or acne-prone skin. In particular, porphyrins—metabolic byproducts produced by Cutibacterium acnes (formerly Propionibacterium acnes)—are known to fluoresce under near-UV excitation (400-410 nm), emitting red light in the 600-650 nm range. The image acquisition module 101 may be equipped with an appropriate light source and emission filters to capture this fluorescence. When red fluorescence is detected in localized areas of the skin, the analysis module 102 may infer the presence of acne-related bacterial activity. In one embodiment, the analysis module 102 may apply a pixel-wise intensity threshold to isolate high-fluorescence regions, which are then flagged as risk areas. In another embodiment, ML based algorithms may be trained on annotated fluorescent images to classify and segment acne lesions. The ML based algorithms may output binary masks or multi-class segmentation maps distinguishing pustules, nodules, comedones, and clear skin.

According to some embodiments, ML based algorithms may be trained to detect inflamed or acne-affected regions using labeled datasets. The analysis module 102 may utilize deep learning models such as UNET, ResNet, EfficientNet, or YOLO to perform object detection and semantic segmentation of risk areas from visible light RGB or multispectral images. These models may be trained to output bounding boxes or pixel-level masks for different skin conditions. For example, the model may output class labels such as “acne pustule,” “inflammatory patch,” or “normal skin.” In scenarios where multiple imaging modalities are available, multimodal fusion architectures may be employed to combine inputs from RGB, infrared, and fluorescence imaging, thereby enhancing the robustness of the detection process.

Without wishing to be bound by theory, other methods of detecting skin risk areas may be used without departing from the spirit or scope of the present invention. For example, thermal imaging may be used to detect localized increases in skin temperature, which can serve as an indicator of inflammation or infection. Similarly, hyperspectral imaging may be used to analyze subtle biochemical differences in skin tissue that correlate with underlying pathology. The analysis module 102 may also utilize temporal analysis—such as monitoring the persistence of redness over time—to differentiate between transient erythema and sustained inflammation. These methods may be combined or adapted depending on the available hardware and intended use environment of the treatment device 100.

Once potential risk areas have been identified, the analysis module 102 may evaluate the severity of the underlying condition using quantitative metrics—such as an inflammation score, or a depression score, as disclosed elsewhere in the present invention. Rather than categorically excluding all risk areas from treatment, the analysis module 102 may apply threshold-based decision rules to determine whether a given area should be excluded or included in the personalized treatment plan. Specifically, if the severity score exceeds a threshold, the area may be classified as a contraindicated area and excluded from treatment. Otherwise, the area may remain eligible for treatment, albeit with precautionary modifications.

As an exemplary embodiment, an inflammation score I may be calculated, prior to the commencement of the treatment, to assess the severity of inflammation:

$$I = aA + b S$$

where A denotes the area within which skin inflammation is detected, S denotes the inflammation severity, and a and b are the weighting factors for area and severity, respectively. The methods for determining A and S may be similar to those previously disclosed in the present invention for calculating the skin redness score R. A threshold value of inflammation score, Iₜ, may be defined. When the analysis module 102 determines that I<Iₜ, the inflammation may be labeled as a mild condition, allowing treatment to proceed with appropriate precautions. Conversely, when I>Iₜ, the area may be classified as a contraindicated area and excluded from the personalized treatment plan.

In the case of a contraindicated area, the analysis module 102 may instruct the UI module 104 to communicate the exclusion to the operator in an intuitive and informative manner. For example, the UI module 104 may display a digital map of the treatment area, with contraindicated areas visually highlighted in a distinct color such as red. Tooltips or textual overlays may be provided to explain the rationale for exclusion (e.g., “Inflamed region—treatment not recommended”). In some embodiments, the UI module 104 may support advanced interaction features, such as zooming into the affected region, toggling between imaging layers (e.g., fluorescence vs. RGB), or displaying classification metadata. Manual confirmation or override functionality may also be available, particularly in professional settings where clinicians can review and, if necessary, adjust the system-generated exclusions. In home-use settings, the UI module 104 may issue a warning and request user confirmation before allowing treatment to proceed over flagged areas. By incorporating these visual feedback and decision support, the system helps prevent inappropriate treatment and promotes user understanding and confidence.

In some embodiments of the present invention, risk areas that do not exceed the threshold score Iₜ may still influence treatment planning. For instance, in RF facial treatments, mild skin redness or low-grade inflammation may not warrant full exclusion but still pose a risk of discomfort or adverse effects if not properly managed. In such cases, the analysis module 102 may analyze the degree of inflammation and, upon determining that I<Iₜ, allow treatment to proceed with specific cautionary instructions. These may include, but are not limited to: (1) instructing the operator to apply a sufficient amount of coupling gel to reduce surface irritation; (2) advising the application of gentler force to minimize mechanical stress on sensitive areas; and (3) automatically reducing the treatment intensity level (e.g., RF output power) when the analysis module 102 detects that the treatment is being applied over the mildly inflamed region. These personalized modifications enable the continuation of treatment while minimizing the risk of exacerbating borderline skin conditions, thereby achieving a balance between therapeutic efficacy and patient safety.

In some embodiments, method of generating a personalized treatment plan for an energy-based aesthetic treatment is provided. The method may be implemented using treatment device 100. In some embodiments, the method may further include obtaining image data identifying a treatment area and a tissue-contacting part of the treatment device. In some embodiments, the method may further include generating a three-dimensional anatomical model of the treatment area based on the image data. In some embodiments, the method may further include identifying one or more risk areas within the treatment area based on the three-dimensional anatomical model and one or more predetermined risk criteria, each risk area corresponding to a region of tissue exhibiting a characteristic selected from: (i) a sunken contour indicative of localized volume loss; (ii) a skin condition indicative of inflammation or infection; or (iii) a combination thereof. In some embodiments, the method may further include computing, for each identified risk area, a severity score using a combination of at least two measurable parameters corresponding to that risk area. In some embodiments, the method may further include classifying each risk area as either a contraindicated area or a mild-risk area based on a comparison of the severity score to a predefined threshold. In some embodiments, the method may further include generating a personalized treatment plan that excludes contraindicated areas from energy delivery and modifies one or more treatment parameters for mild-risk areas. In some embodiments, the method may further include presenting, by a user interface module 104 of the treatment device 100, a visual representation of the personalized treatment plan, where the personalized treatment plan identifies excluded areas and modified treatment zones, to an operator.

In some embodiments, the depression volume is computed by calculating a volume differential between the user's anatomical model and the reference model in a region of interest.

In some embodiments, identifying the one or more risk areas comprises detecting inflammation based on one or more of: skin redness, increased vascularization, elevated skin temperature, or fluorescence of porphyrins.

In some embodiments, the severity score for an inflamed region is calculated as a weighted sum of an inflammation area and an inflammation severity.

In some embodiments, the user interface module presents the personalized treatment plan by visually highlighting contraindicated areas with a first color and mild-risk areas with a second, different color.

In some embodiments, the personalized treatment plan includes modifying a treatment intensity level or electrode configuration for mild-risk areas.

The foregoing disclosure provides illustration and description but is not intended to be exhaustive or to limit the embodiments to the precise form disclosed. Modifications and variations may be made in light of the above disclosure or may be acquired from practice of the embodiments.

As used herein, the term “module” is intended to be broadly construed as hardware, software, and/or a combination of hardware and software.

Some embodiments are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc., depending on the context.

Certain user interfaces have been described herein. A user interface may include a graphical user interface, a non-graphical user interface, a text-based user interface, etc. A user interface may provide information for display. In some embodiments, a user may interact with the information, such as by providing input via an input component of a device that provides the user interface for display. In some embodiments, a user interface may be configurable by a device and/or a user (e.g., a user may change the size of the user interface, information provided via the user interface, a position of information provided via the user interface, etc.). Additionally, or alternatively, a user interface may be pre-configured to a standard configuration, a specific configuration based on a type of device on which the user interface is displayed, and/or a set of configurations based on capabilities and/or specifications associated with a device on which the user interface is displayed.

No element, act, or instruction used herein should be construed as critical or essential unless explicitly described as such. Also, as used herein, the articles “a” and “an” are intended to include one or more items and may be used interchangeably with “one or more.” Furthermore, as used herein, the term “set” is intended to include one or more items (e.g., related items, unrelated items, a combination of related and unrelated items, etc.), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise

What is claimed is:

1. A method of customizing an energy-based aesthetic treatment, comprising:
obtaining, by a treatment device, image data identifying a treatment area and a tissue-contacting part of the treatment device;
selecting, by the treatment device, an anatomical classification criterion based on a type of energy modality used for the energy-based aesthetic treatment, the anatomical classification criterion being an anatomical characteristic with a measurable or observable property that differs across regions of tissue in the treatment area;
determining, by the treatment device and by analyzing the image data using the anatomical classification criterion, a plurality of treatment sub-areas within the treatment area, the treatment sub-areas being differentiated based on the measurable or observable property of the anatomical characteristic which varies across the regions of tissue in the treatment area;
while the energy-based aesthetic treatment is being performed, determining, by the treatment device, a current treatment sub-area of the treatment area that is actively being treated or targeted for treatment, the current treatment sub-area being determined based on a position of the tissue-contacting part of the treatment device relative to one of the plurality of treatment sub-areas; and
adjusting, by the treatment device, one or more treatment settings during the energy-based aesthetic treatment based on the measurable or observable property of the anatomic characteristic of a region of tissue in the current treatment sub-area.

2. The method of claim 1, further comprising:
determining that the tissue-contacting part of the treatment device at least partially overlaps with a risk area; and
performing one or more corrective actions including at least one of:
adjusting at least one treatment setting of the one or more treatment settings, and
generating and providing a warning or recommendation for display on a user interface accessible by an operator of the treatment device.

3. The method of claim 1, further comprising:
determining that the tissue-contacting part of the treatment device is outside of an intended treatment sub-area; and
performing one or more corrective actions including at least one of:
adjusting at least one treatment setting of the one or more treatment settings, and
generating and providing a warning or recommendation for display on a user interface accessible by an operator of the treatment device.

4. The method of claim 1, wherein the anatomical classification criterion is one of:
a skin thickness,
a depth of a bone relative to a skin surface,
a depth of a muscle or a nerve fiber relative to the skin surface,
an orientation of the muscle or the nerve fiber, or
a skin condition.

5. The method of claim 1, wherein the anatomical classification criterion selected is a skin thickness, wherein the plurality of treatment sub-areas are determined to be regions of tissue containing different skin thicknesses, and wherein adjusting the one or more treatment settings comprises:
adjusting a treatment intensity level based on the skin thickness identified in the region of tissue included in the current treatment sub-area.

6. The method of claim 1, wherein the anatomical classification criterion selected is a depth of a bone relative to a skin surface, wherein the plurality of treatment sub-areas are determined to be regions of tissue containing bones located at different depths relative to the skin surface, and wherein adjusting the one or more treatment settings comprises:
adjusting a focal depth level based on the depth of the bone in the region of tissue included in the current treatment sub-area.

7. The method of claim 1, wherein the anatomical classification criterion selected is a depth of a muscle or a nerve fiber relative to a skin surface, wherein the plurality of treatment sub-areas are determined to be regions of tissue containing muscles or nerve fibers located at different depths, and wherein adjusting the one or more treatment settings comprises:
adjusting an amplitude of an electrical waveform based on the depth of the muscle or the nerve fiber in the region of tissue included in the current treatment sub-area.

8. The method of claim 1, wherein the anatomical classification criterion selected is an orientation of a muscle or a nerve fiber, wherein the plurality of treatment sub-areas are determined to be regions of tissue containing muscles or nerve fibers oriented in different directions, and wherein adjusting the one or more treatment settings comprises:
adjusting a direction of electrical current delivery to be aligned or substantially aligned with the orientation of the muscle or the nerve fiber.

9. The method of claim 1, wherein the anatomical classification criterion selected is a skin condition, wherein the plurality of treatment sub-areas are determined to be regions of tissue corresponding to different skin conditions, and wherein adjusting the one or more treatment settings comprises:
adjusting one or more light wavelength settings based on the skin condition found in the region of tissue included in the current treatment sub-area.

10. The method of claim 1, wherein the treatment device is configured to deliver at least two different types of energy, wherein selecting the anatomical classification criterion comprises:
selecting the anatomical classification criterion based on a first type of energy modality used for the energy-based aesthetic treatment, and
further selecting another anatomical classification criterion based on a second type of energy modality used for the energy-based aesthetic treatment;
wherein determining the plurality of treatment sub-areas comprises:
determining a first set of treatment sub-areas as regions of tissue containing the measurable or observable property of the anatomical classification criterion, and
determining a second set of treatment sub-areas as regions of tissue containing a measurable or observable property of the other anatomical classification criterion; and
wherein the method further comprises:
delivering a first type of energy, a second type of energy, or both the first and second type of energy, at a moment in time, based on whether the tissue-contacting part of the treatment device is within one or both of: the first set of treatment sub-areas, and the second set of treatment sub-areas.

11. A treatment device, comprising:
one or more cameras configured to capture image data identifying a treatment area and a tissue-contacting part of the treatment device;
an energy emission component configured to deliver energy as part of an energy-based aesthetic treatment;
one or more memories; and
one or more processors, communicatively coupled to the one or more cameras and operatively coupled to the energy emission component and the one or more memories, to:
receive the image data from the one or more cameras;
select an anatomical classification criterion based on a type of energy modality used for the energy-based aesthetic treatment, the anatomical classification criterion being an anatomical characteristic with a measurable or observable property that differs across regions of tissue in the treatment area;
determine, by analyzing the image data using the anatomical classification criterion, a plurality of treatment sub-areas within the treatment area, the treatment sub-areas being differentiated based on the measurable or observable property of the anatomical characteristic which varies across the regions of tissue in the treatment area;
while the energy-based aesthetic treatment is being performed, determine a current treatment sub-area of the treatment area that is actively being treated or targeted for treatment, the current treatment sub-area being determined based on a position of the tissue-contacting part of the treatment device relative to one of the plurality of treatment sub-areas; and
adjust one or more treatment settings during the energy-based aesthetic treatment based on the measurable or observable property of the anatomic characteristic of a region of tissue in the current treatment sub-area, where adjustment of the one or more treatment settings influences the energy delivered by the energy emission component.

12. The treatment device of claim 11, wherein the one or more processors are further to:
determine that the treatment area contains a risk area;
determine that the current treatment sub-area is within the risk area; and
perform one or more corrective actions including at least one of:
adjust at least one treatment setting of the one or more treatment settings, and
generate and provide a warning or recommendation for display on a user interface accessible by an operator of the treatment device.

13. The treatment device of claim 11, wherein the one or more processors are further to:
determine that the current treatment sub-area is outside of an intended treatment sub-area; and
perform one or more corrective actions including at least one of:
adjust at least one treatment setting of the one or more treatment settings, and
generate and provide a warning or recommendation for display on a user interface accessible by an operator of the treatment device.

14. The treatment device of claim 11, wherein the anatomical classification criterion is one of:
a skin thickness,
a depth of a bone relative to a skin surface,
a depth of a muscle or a nerve fiber relative to the skin surface,
an orientation of the muscle or the nerve fiber, or
a skin condition.

15. The treatment device of claim 11, wherein the anatomical classification criterion selected is a skin thickness, wherein the plurality of treatment sub-areas are determined to be regions of tissue containing different skin thicknesses, and wherein the one or more processors, when adjusting the one or more treatment settings, are to:
adjust a treatment intensity level based on the skin thickness identified in the region of tissue included in the current treatment sub-area.

16. The treatment device of claim 11, wherein the anatomical classification criterion selected is a depth of a bone relative to a skin surface, wherein the plurality of treatment sub-areas are determined to be regions of tissue containing bones located at different depths relative to the skin surface, and wherein the one or more processors, when adjusting the one or more treatment settings, are to:
adjust a focal depth level based on the depth of the bone in the region of tissue included in the current treatment sub-area.

17. The treatment device of claim 11, wherein the anatomical classification criterion selected is a depth of a muscle or a nerve fiber relative to a skin surface, wherein the plurality of treatment sub-areas are determined to be regions of tissue containing muscles or nerve fibers located at different depths, wherein the one or more processors, when adjusting the one or more treatment settings, are to:
adjust an amplitude of an electrical waveform based on the depth of the muscle or the nerve fiber in the region of tissue included in the current treatment sub-area.

18. The treatment device of claim 11, wherein the anatomical classification criterion selected is an orientation of a muscle or a nerve fiber relative to a known reference orientation, wherein the plurality of treatment sub-areas are determined to be regions of tissue containing muscles or nerve fibers oriented in different directions, and wherein the one or more processors, when adjusting the one or more treatment settings, are to:
adjust a direction of electrical current delivery to be aligned or substantially aligned with the orientation of the muscle or the nerve fiber.

19. The treatment device of claim 11, wherein the anatomical classification criterion selected is a skin condition, wherein the plurality of treatment sub-areas are determined to be regions of tissue corresponding to different skin conditions, and wherein the one or more processors, when adjusting the one or more treatment settings, are to:
adjust one or more light wavelength settings based on the skin condition found in the region of tissue included in the current treatment sub-area.

20. The treatment device of claim 11, wherein the treatment device is configured to deliver at least two different types of energy, wherein the one or more processors, when selecting the anatomical classification criterion, are to:
select the anatomical classification criterion based on a first type of energy modality used for the energy-based aesthetic treatment, and
select another anatomical classification criterion based on a second type of energy modality used for the energy-based aesthetic treatment;
wherein the one or more processors, when determining the plurality of treatment sub-areas, are to:
determine a first set of treatment sub-areas as regions of tissue containing the measurable or observable property of the anatomical classification criterion, and
determine a second set of treatment sub-areas as regions of tissue containing a measurable or observable property of the other anatomical classification criterion; and
wherein the one or more processors are further to:
deliver a first type of energy, a second type of energy, or both the first and second type of energy, at a moment in time, based on whether the tissue-contacting part of the treatment device is within one or both of: the first set of treatment sub-areas and the second set of treatment sub-areas.
